(19) United States
(12) Patent Application Publication
(10) Pub. No.: US 2008/0146602 A1
(43) Pub. Date: Jun. 19, 2008

(54) Thienopyridine Derivatives, Production Method and Use Thereof

(75) Inventors: Atsuo Baba, Tsukuba-shi (JP); Tsuneo Yasuma, Osaka-shi (JP); Hiroyuki Tawada, Takatsuki-shi (JP); Makoto Yamashita, Osaka-shi (JP)

Correspondence Address: EDWARDS ANGELL PALMER & DODGE LLP, P.O. BOX 55874, BOSTON, MA, 02205, US

(73) Assignee: TAKEDA PHARMACEUTICAL COMPANY LIMITED, Osaka-shi (JP)

(21) Appl. No.: 11/596,634

(22) PCT Filed: May 13, 2005
(86) PCT No.: PCT/JP05/09205

§ 371 Date: Nov. 13, 2006

(30) Foreign Application Priority Data
May 14, 2004 (JP) 2004-144659
Mar. 15, 2005 (JP) 2005-073745

(51) Int. Cl. A61K 31/4365; C07D 471/04; A61P 19/02

(52) U.S. Cl. 514/291; 546/80

(57) ABSTRACT

A compound represented by the formula (I)

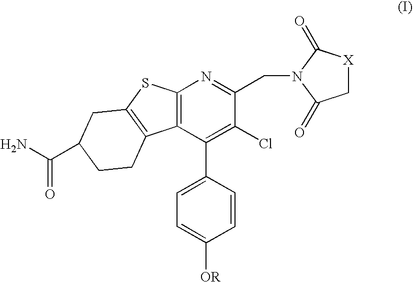

wherein R is a hydrogen atom or a C₁₋₄ alkyl group, and X is CH₂, O or S, or a salt thereof. The present invention provides a novel thienopyridine derivative having an anti-inflammatory effect, a bone resorption suppressing effect, an immune cytokine production suppressing effect and the like, and useful as a pharmaceutical agent such as an agent for the prophylaxis or treatment of arthritis such as rheumatoid arthritis and the like, and the like, and the like.

### TECHNICAL FIELD

The present invention relates to a novel thienopyridine derivative having an anti-inflammatory activity, a bone resorption-suppressing activity, an immune cytokine production-suppressing activity and the like, which is useful as a pharmaceutical agent such as an agent for the prophylaxis or treatment and the like of arthritis such as rheumatoid arthritis and the like, and the like, a production method thereof and use thereof.

### BACKGROUND ART

Arthritis is an inflammatory disease of joint, which includes, as major diseases, rheumatoid-like arthritis and related diseases thereof associated with inflammation in the joint.

Particularly, rheumatoid-like arthritis is also called rheumatoid arthritis, which is chronic polyarthritis having, as major lesions, inflammatory changes in the synovium in the joint internal capsule layer. Arthritis such as rheumatoid-like arthritis and the like is progressive, shows joint disorders such as deformation, tetany and the like of joint and when aggravated without effective treatment, often resulting in severe physical handicap.

As conventional treatment of these arthritises, steroids such as adrenocortical hormone and the like (e.g., cortisone etc.), nonsteroidal anti-inflammatory agents (e.g., aspirin, piroxicam, indomethacin etc.), gold compounds (e.g., gold thiomalate etc.), antirheumatic drugs (e.g., chloroquine formulation, D-penicillamine etc.), antipodagric drugs (e.g., colchicine etc.), immunosuppressants (e.g., cyclophosphamide, azathiopurine, methotrexate, levamisole etc.) and the like have been used for a drug therapy. However, some of these pharmaceutical agents have shown problems of side effects which are severe or which render long-term use difficult, insufficient effect, or ineffectiveness for the already developed arthritis, and the like.

Thienopyridine derivatives useful as anti-inflammatory agents or therapeutic agents for rheumatoid arthritis have been reported in WO 01/64685 and the like.

### DISCLOSURE OF THE INVENTION

In clinical cases of arthritis and the like, a more superior pharmaceutical agent for the prophylaxis or treatment of arthritis and the like has still been desired.

The present inventors have conducted intensive studies in view of the above-mentioned situation and found that a thienopyridine derivative represented by the following formula (I) has a potent anti-inflammatory activity, particularly, an antiarthritic activity, based on its specific chemical structure, and is useful as a joint destruction suppressing agent, and as a bone resorption suppressing agent because it has a superior bone resorption suppressing activity directly on the bone, and further as an immunosuppressant, which resulted in the completion of the present invention.

Accordingly, the present invention relates to [1] a compound represented by the formula (I)

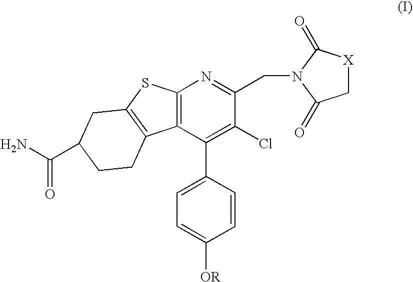

wherein R is a hydrogen atom or a C₁₋₄ alkyl group and X is CH₂, O or S (hereinafter simply referred to as compound (I)) or a salt thereof,
[2] 3-chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxamide or a salt thereof,
3-chloro-2-[(2,5-dioxo-1-pyrrolidinyl)methyl]-5,6,7,8-tetrahydro-4-(4-methoxyphenyl)[1]benzothieno[2,3-b]pyridine-7-carboxamide or a salt thereof, or
3-chloro-2-[(2,4-dioxo-1,3-thiazolidin-3-yl)methyl]-5,6,7,8-tetrahydro-4-(4-methoxyphenyl)[1]benzothieno[2,3-b]pyridine-7-carboxamide, or a salt thereof,
   [3] a prodrug of the compound of the aforementioned [1],
   [4] a production method of an optically active compound represented by the formula

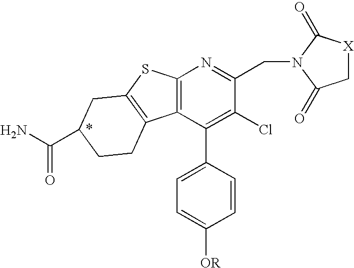

wherein R is a hydrogen atom or a C₁₋₄ alkyl group, X is CH₂, O or S and * shows the position of the optically active asymmetric carbon, or a salt thereof,
which comprises cleaving an amino-protecting group of an optically active compound represented by the formula

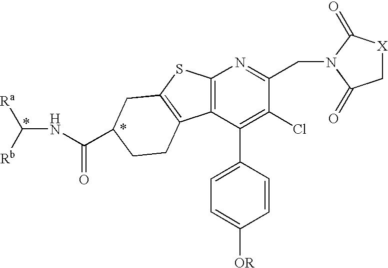

wherein R^{a} is an aryl group optionally having substituents, R^{b} is an alkyl group optionally having substituents or an aryl group optionally having substituents, which is different from R^{a}, and other symbols are as defined above, or a salt thereof,
[5] the production method of the aforementioned [4], wherein R^{a} is a phenyl group optionally having substituents, R^{b} is an alkyl group optionally having substituents or a phenyl group optionally having substituents, which is different from R^{a},
[6] a production method of an optically active compound represented by the formula

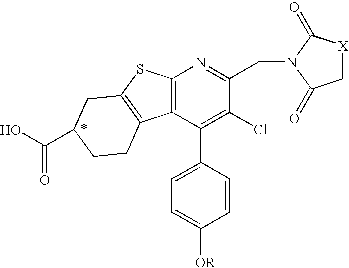

wherein R is a hydrogen atom or a C₁₋₄ alkyl group, X is CH₂, O or S and * shows the position of the optically active asymmetric carbon, or a salt thereof,
which comprises hydrolyzing an optically active compound represented by the formula

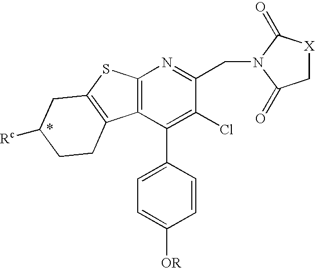

wherein R^{c} is an ester group having an optically active asymmetric carbon, and other symbols are as defined above, or a salt thereof,
[7] a production method of an optically active compound represented by the formula

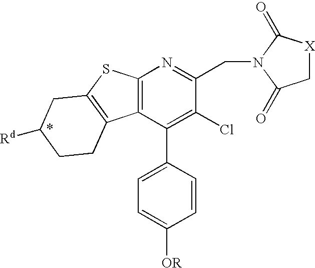

wherein R is a hydrogen atom or a C₁₋₄ alkyl group, R^{d} is an esterified or amidated carboxyl group having an optically active asymmetric carbon, X is CH₂, O or S, and * shows the position of the optically active asymmetric carbon, or a salt thereof, which comprises reacting a compound represented by the formula

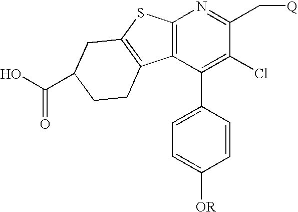

wherein R is as defined above and Q is a leaving group or a group represented by the formula

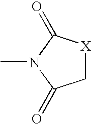

wherein X is as defined above, or a salt thereof, with an amine compound having an optically active asymmetric carbon or an alcohol compound having an optically active asymmetric carbon, or a salt thereof to give a diastereomer mixture or a salt thereof, optically resolving the diastereomer mixture or a salt thereof, and when Q is a leaving group, further reacting with a compound represented by the formula

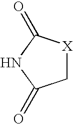

wherein X is as defined above, or a salt thereof,
[8] a pharmaceutical agent comprising the compound of the aforementioned [1] or a prodrug thereof,
[9] the pharmaceutical agent of the aforementioned [8], which is a T cell differentiation modulating agent,
[10] the pharmaceutical agent of the aforementioned [8], which is an agent for the prophylaxis or treatment of inflammatory diseases,
[11] the pharmaceutical agent of the aforementioned [8], which is an agent for the prophylaxis or treatment of immune diseases,
[12] the pharmaceutical agent of the aforementioned [8], which is an agent for the prophylaxis or treatment of rheumatoid arthritis,
[13] a method for preventing or treating rheumatoid arthritis, which comprises administering an effective amount of the compound of the aforementioned [1] or a prodrug thereof to a mammal, and
[14] use of the compound of the aforementioned [1] or a prodrug thereof for the production of an agent for the prophylaxis or treatment of rheumatoid arthritis.

Furthermore, the present invention also relates to

[15] a compound represented by the formula

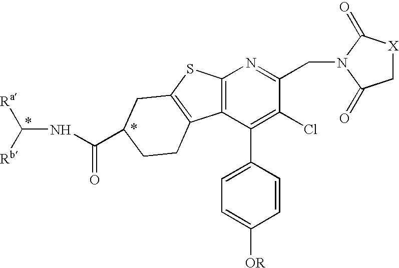

wherein R is a hydrogen atom or a C₁₋₄ alkyl group, R^{a′} is an aryl group optionally having substituents, R^{b′} is an alkyl group optionally having substituents, X is CH₂, O or S, and * shows the position of the optically active asymmetric carbon, or a salt thereof,
[16] a compound represented by the formula

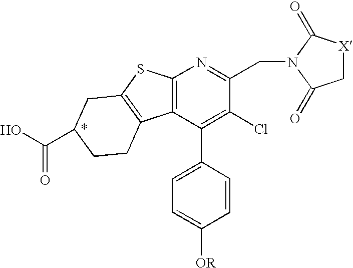

wherein R is a hydrogen atom or a C₁₋₄ alkyl group, X′ is O or S, and * shows the position of the optically active asymmetric carbon, or a salt thereof, and
[17] a compound represented by the formula

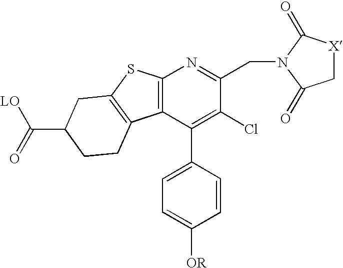

wherein R is a hydrogen atom or a C₁₋₄ alkyl group, X′ is O or S, and L is a C₁₋₄ alkyl group, or a salt thereof.

### BEST MODE FOR EMBODYING THE INVENTION

The definition of each symbol of the compounds described in the present specification is explained in the following.

In the aforementioned formulas, R is a hydrogen atom or a C₁₋₄ alkyl group (methyl, ethyl, propyl, isopropyl, butyl, isobutyl, sec-butyl, tert-butyl). As R, a C₁₋₄ alkyl group such as methyl and the like is preferable.

In the aforementioned formulas, X is CH₂, O or S.

In the aforementioned formulas, X′ is O or S.

In the aforementioned formulas, * shows the position of the optically active asymmetric carbon. In the description of the present specification, the “optically active” in the “optically active compound” and the like means containing at least one optically active asymmetric carbon, and often used to mean the same as “having an optically active asymmetric carbon”.

In the aforementioned formulas, as the “leaving group” when Q is a “leaving group”, for example, a halogen atom (e.g., fluorine, chlorine, bromine, iodine etc.), an alkylsulfonyl group (e.g., a C₁₋₆ alkylsulfonyl group such as methanesulfonyl, ethanesulfonyl and the like, etc.), an optionally halogenated alkylsulfonyloxy group (e.g., an optionally halogenated C₁₋₆ alkylsulfonyloxy group such as methanesulfonyloxy, ethanesulfonyloxy, trichloromethanesulfonyloxy and the like, etc.), an optionally substituted arylsulfonyloxy group (e.g., an optionally substituted benzenesulfonyloxy group such as p-toluenesulfonyloxy, benzenesulfonyloxy and the like, etc.) and the like can be mentioned, with preference given to a halogen atom (e.g., fluorine, chlorine, bromine, iodine etc.).

In the aforementioned formulas, as the “aryl group” of the “aryl group optionally having substituents” for R^{a} and/or R^{b}, for example, a C₆₋₁₄ aryl group (e.g., phenyl, 1-naphthyl, 2-naphthyl, biphenylyl, 2-anthryl etc.) can be mentioned, with preference given to phenyl, 1-naphthyl and the like, more preferably phenyl.

As the “substituent” of the “aryl group optionally having substituents” for R^{a} and/or R^{b}, 1 to 3 substituents selected from

(1) a halogen atom (e.g., fluorine, chlorine, bromine, iodine etc.);
(2) nitro;
(3) cyano;
(4) C₁₋₆ alkyl optionally having 1 to 3 halogen atoms (e.g., methyl, chloromethyl, difluoromethyl, trichloromethyl, trifluoromethyl, ethyl, 2-bromomethyl, 2,2,2-trifluoroethyl, pentafluoroethyl, propyl, 3,3,3-trifluoropropyl, isopropyl, butyl, 4,4,4-trifluorobutyl, isobutyl, sec-butyl, t-butyl, pentyl, isopentyl, neopentyl, 5,5,5-trifluoropentyl, hexyl, 6,6,6-trifluorohexyl etc.);
(5) C₆₋₁₄ aryl (e.g., phenyl, 1-naphthyl, 2-naphthyl, biphenylyl, 2-anthryl etc.);
(6) hydroxy;
(7) C₁₋₆ alkoxy (e.g., methoxy, ethoxy, propoxy, isopropoxy, butoxy, isobutoxy, sec-butoxy, pentyloxy, hexyloxy etc.);
(8) C₆₋₁₄ aryloxy (e.g., phenyloxy, naphthyloxy etc.);
(9) mercapto;
(10) C₁₋₆ alkylthio optionally having 1 to 3 halogen atoms (e.g., methylthio, difluoromethylthio, trifluoromethylthio, ethylthio, propylthio, isopropylthio, butylthio, 4,4,4-trifluorobutylthio, pentylthio, hexylthio etc.);
(11) C₆₋₁₄ arylthio (e.g., phenylthio, naphthylthio etc.);
(12) amino;
(13) mono-C₁₋₆ alkylamino (e.g., methylamino, ethylamino etc.);
(14) mono-C₆₋₁₄ arylamino (e.g., phenylamino, 1-naphthylamino, 2-naphthylamino etc.);
(15) di-C₁₋₆ alkylamino (e.g., dimethylamino, diethylamino etc.);
(16) di-C₆₋₁₄ arylamino (e.g., diphenylamino etc.)
(17) formyl;
(18) C₁₋₆ alkyl-carbonyl (e.g., acetyl, propionyl etc.);
(19) C₆₋₁₄ aryl-carbonyl (e.g., benzoyl, 1-naphthoyl, 2-naphthoyl etc.);
(20) carboxyl;
(21) C₁₋₆ alkoxy-carbonyl (e.g., methoxycarbonyl, ethoxycarbonyl, propoxycarbonyl, tert-butoxycarbonyl etc.);
(22) C₆₋₁₄ aryloxy-carbonyl (e.g., phenoxycarbonyl etc.);
(23) carbamoyl;
(24) thiocarbamoyl;
(25) mono-C₁₋₆ alkyl-carbamoyl (e.g., methylcarbamoyl, ethylcarbamoyl etc.);
(26) di-C₁₋₆ alkyl-carbamoyl (e.g., dimethylcarbamoyl, diethylcarbamoyl, ethylmethylcarbamoyl etc.);
(27) C₆₋₁₄ aryl-carbamoyl (e.g., phenylcarbamoyl, 1-naphthylcarbamoyl, 2-naphthylcarbamoyl etc.);
(28) C₁₋₆ alkylsulfonyl (e.g., methylsulfonyl, ethylsulfonyl etc.);
(29) C₆₋₁₄ arylsulfonyl (e.g., phenylsulfonyl, 1-naphthylsulfonyl, 2-naphthylsulfonyl etc.);
(30) C₁₋₆ alkylsulfinyl (e.g., methylsulfinyl, ethylsulfinyl etc.);
(31) C₆₋₁₄ arylsulfinyl (e.g., phenylsulfinyl, 1-naphthylsulfinyl, 2-naphthylsulfinyl etc.);
(32) formylamino;
(33) C₁₋₆ alkyl-carbonylamino (e.g., acetylamino etc.);
(34) C₆₋₁₄ aryl-carbonylamino (e.g., benzoylamino, naphthoylamino etc.);
(35) C₁₋₆ alkoxy-carbonylamino (e.g., methoxycarbonylamino, ethoxycarbonylamino, propoxycarbonylamino, butoxycarbonylamino etc.);
(36) C₁₋₆ alkylsulfonylamino (e.g., methylsulfonylamino, ethylsulfonylamino etc.);
(37) C₆₋₁₄ arylsulfonylamino (e.g., phenylsulfonylamino, 2-naphthylsulfonylamino, 1-naphthylsulfonylamino etc.);
(38) C₁₋₆ alkyl-carbonyloxy (e.g., acetoxy, propionyloxy etc.);
(39) C₆₋₁₄ aryl-carbonyloxy (e.g., benzoyloxy, naphthylcarbonyloxy etc.);
(40) C₁₋₆ alkoxy-carbonyloxy (e.g., methoxycarbonyloxy, ethoxycarbonyloxy, propoxycarbonyloxy, butoxycarbonyloxy etc.),
(41) mono-C₁₋₆ alkyl-carbamoyloxy (e.g., methylcarbamoyloxy, ethylcarbamoyloxy etc.);
(42) di-C₁₋₆ alkyl-carbamoyloxy (e.g., dimethylcarbamoyloxy, diethylcarbamoyloxy etc.);
(43) C₆₋₁₄ aryl-carbamoyloxy (e.g., phenylcarbamoyloxy, naphthylcarbamoyloxy etc.);
(44) 5 to 7-membered saturated cyclic amino optionally containing one nitrogen atom and, besides carbon atoms, 1 or 2 kinds of 1 to 4 hetero atoms selected from nitrogen atom, sulfur atom and oxygen atom (e.g., pyrrolidin-1-yl, piperidino, piperazin-1-yl, morpholino, thiomorpholino, hexahydroazepin-1-yl etc.);
(45) a 5- to 10-membered aromatic heterocyclic group containing, besides carbon atoms, 1 or 2 kinds of 1 to 4 hetero atoms selected from nitrogen atom, sulfur atom and oxygen atom (e.g., 2-thienyl, 3-thienyl, 2-pyridyl, 3-pyridyl, 4-pyridyl, 2-quinolyl, 3-quinolyl, 4-quinolyl, 5-quinolyl, 8-quinolyl, 1-isoquinolyl, 3-isoquinolyl, 4-isoquinolyl, 5-isoquinolyl, 1-indolyl, 2-indolyl, 3-indolyl, 2-benzothiazolyl, 2-benzo[b]thienyl, 3-benzo[b]thienyl, 2-benzo[b]furanyl, 3-benzo[b]furanyl etc.);
(46) C₁₋₃ alkylenedioxy (e.g., methylenedioxy, ethylenedioxy etc.) and the like.

As the “alkyl group” of the “alkyl group optionally having substituents” for R^{b}, a C₁₋₆ alkyl group (e.g., methyl, ethyl, propyl, isopropyl, butyl, isobutyl, sec-butyl, tert-butyl, pentyl, hexyl etc.) can be mentioned, and as the “substituent”, 1 to 3 substituents similar to those of the above-mentioned “aryl group optionally having substituents” can be mentioned. Preferably, R^{b} is a C₁₋₆ alkyl group (e.g., methyl etc.).

In the aforementioned formulas, as the “aryl group optionally having substituents” for R^{a′}, for example, those similar to the ones exemplified for the aforementioned “aryl group optionally having substituents” for R^{a} and/or R^{b} can be mentioned, with preference given to a C₆₋₁₄ aryl group (e.g., phenyl etc.).

In the aforementioned formulas, as the “alkyl group optionally having substituents” for R^{b′}, for example, those similar to the ones exemplified for the aforementioned “alkyl group optionally having substituents” for R^{b} can be mentioned, with preference given to a C₁₋₆ alkyl group (e.g., methyl etc.).

In the aforementioned formulas, R^{c} is an ester group having an optically active asymmetric carbon. Specifically, R^{c} is an ester group having an optically active asymmetric carbon, which can be obtained by esterifying a carboxyl group with an alcohol compound having an optically active asymmetric carbon represented by the formula R^{e}—OH.

Here, as the “alcohol compound having an optically active asymmetric carbon” represented by R^{e}—OH, for example, (R)-(+)-1-phenylethanol, (S)-(−)-1-phenylethanol, (R) — (+)-methyl lactate, (S)-(−)-methyl lactate, (S)-(+)-methyl mandelate, (R)-(−)-methyl mandelate, (S)-(+)-benzyl mandelate, (R)-(−)-benzyl mandelate, D-(−)-methyl tartrate, L-(+)-methyl tartrate, D-(−)-pantolactone, L-(+)-pantolactone, (S)-(+)-1-phenyl-1,2-ethanediol-2-tosylate, (R)-(+)-2-bromo-α-methylbenzyl alcohol, L-(−)-dimethyl malate, D-(+)-dimethyl malate, (R)-ethyl 2-hydroxy-4-phenylbutyrate and the like are preferably used, and any kind of compound can be used as long as it is an alcohol compound having an optically active asymmetric carbon.

For example, as described in Step 10 below, these alcohols having an optically active asymmetric carbon is reacted with carboxylic acid compound (VI) to give compound (XII) having an ester group having an optically active asymmetric carbon R^{c}.

In the above-mentioned formulas, of the “esterified or amidated carboxyl group having an optically active asymmetric carbon” for R^{d}, as the “amidated carboxyl group having an optically active asymmetric carbon”, for example, a group formed by amidating a carboxyl group with an amine compound having an optically active asymmetric carbon such as (S)-(−)-α-methylbenzylamine, (R)-(+)-α-methylbenzylamine, (S)-α-methyl-4-nitrobenzylamine, (R)-α-methyl-4-nitrobenzylamine, (S)-(−)-1-(1-naphthyl)ethylamine, (R)-(+)-1-(1-naphthyl)ethylamine and the like, and the like can be mentioned.

In addition, as the “esterified carboxyl group having an optically active asymmetric carbon” for R^{d}, for example, a group formed by esterifying a carboxyl group with an alcohol compound having an optically active asymmetric carbon explained as the above-mentioned R^{e}—OH and the like can be mentioned.

In Steps 1, 2 and 4 mentioned below, as the “lower alkyl group” for L, for example, those similar to the ones exemplified for the aforementioned “C₁₋₄ alkyl group” for R can be mentioned.

The production method of compound (I) is explained below.

The above-mentioned compound (I) can be produced as follows. That is,

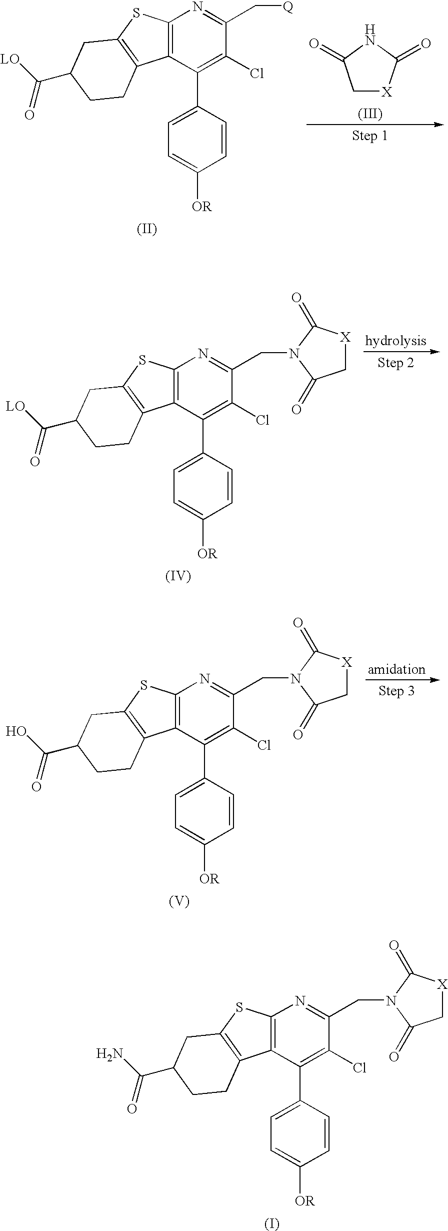

wherein the symbols are as defined above.

### Step 1

In this Step, compound (II) is reacted with compound (III) to give compound (IV).

This reaction is carried out according to a conventional method in the presence of a base in a solvent that does not adversely influence the reaction. As the base, for example, alkali metal salts such as potassium hydroxide, sodium hydroxide, sodium hydrogen carbonate, potassium carbonate and the like; amines such as pyridine, triethylamine, N,N-dimethylaniline, 1,8-diazabicyclo[5.4.0]undeca-7-ene and the like; metal hydrides such as potassium hydride, sodium hydride and the like; alkali metal alkoxides such as sodium methoxide, sodium ethoxide, potassium t.-butoxide, sodium t.-butoxide and the like, and the like can be mentioned.

The amount of the base to be used is preferably 1 to 5 molar equivalents relative to compound (II).

As the solvent that does not adversely influence the reaction, for example, aromatic hydrocarbons such as benzene, toluene, xylene and the like; ethers such as tetrahydrofuran, dioxane, diethyl ether and the like; ketones such as acetone, 2-butanone and the like; halogenated hydrocarbons such as chloroform, dichloromethane and the like; amides such as N,N-dimethylformamide, N,N-dimethylacetamide, 1-methyl-2-pyrrolidone and the like; nitriles such as acetonitrile, propionitrile and the like; sulfoxides such as dimethyl sulfoxide and the like and the like can be mentioned. These solvents may be used in a combination of two or more kinds thereof at appropriate ratios.

The reaction temperature is generally −50 to 150° C., preferably −10 to 100° C.

The reaction time is generally 0.5 to 20 hrs.

### Step 2

In this step, the ester moiety of compound (IV) is hydrolyzed to give carboxylic acid compound (V).

This reaction is generally carried out in the presence of an acid in a solvent that does not adversely influence the reaction.

As the acid, for example, mineral acids such as hydrochloric acid, hydrobromic acid, sulfuric acid, nitric acid, phosphoric acid and the like, organic acids such as acetic acid, propionic acid, trichloroacetic acid, trifluoroacetic acid and the like, sulfonic acids such as methanesulfonic acid, ethanesulfonic acid, trifluoromethanesulfonic acid, benzenesulfonic acid and the like and the like can be mentioned. The amount of the acid to be used is generally 0.1 to 200 molar equivalents, preferably 1 to 100 molar equivalents, relative to compound (IV). The acid may be used as a solvent, two or more kinds thereof may be used in combination at appropriate ratios.

The reaction temperature is generally −10 to 200° C., preferably 0 to 150° C.

The reaction time is generally 0.5 to 20 hrs.

As the solvent that does not adversely influence the reaction, for example, alcohols such as methanol, ethanol, propanol, isopropanol, methoxyethanol and the like; ethers such as dioxane, dimethoxyethane and the like; ketones such as acetone, 2-butanone and the like; amides such as N,N-dimethylformamide, N,N-dimethylacetamide, 1-methyl-2-pyrrolidone and the like; sulfoxides such as dimethyl sulfoxide and the like; water and the like can be mentioned. These solvents may be used in a combination of two or more kinds thereof at appropriate ratios.

Alternatively, this reaction may be carried out in the presence of a base in a solvent that does not adversely influence the reaction.

As the base, for example, alkali metal salts such as potassium hydroxide, sodium hydroxide, sodium hydrogen carbonate, potassium carbonate and the like can be mentioned. The amount of the base to be used is 0.1 to 100 molar equivalents, preferably 1 to 20 molar equivalents, relative to compound (IV).

As the solvent that does not adversely influence the reaction, for example, aromatic hydrocarbons such as benzene, toluene, xylene and the like; ethers such as tetrahydrofuran, dioxane, diethyl ether and the like; halogenated hydrocarbons such as chloroform, dichloromethane and the like; ketones such as acetone, 2-butanone and the like; amides such as N,N-dimethylformamide, N,N-dimethylacetamide, 1-methyl-2-pyrrolidone and the like; nitriles such as acetonitrile, propionitrile and the like; sulfoxides such as dimethyl sulfoxide and the like; water and the like can be mentioned. These solvents may be used in a combination of two or more kinds thereof at appropriate ratios.

The reaction temperature is generally −10 to 150° C., preferably 0 to 110° C.

The reaction time is generally 0.5 to 20 hrs.

### Step 3

In this step, carboxylic acid compound (V) is condensed with ammonia to give compound (I).

This reaction is carried out by, for example, a method comprising directly condensing compound (V) with ammonia using a condensing agent, or a method comprising reacting a reactive derivative of compound (V) with ammonia as appropriate and the like.

As the condensing agent, for example, carbodiimide condensing agents such as dicyclohexylcarbodiimide, diisopropylcarbodiimide, 1-ethyl-3-dimethylaminopropylcarbodiimide and hydrochloride thereof and the like; phosphate condensing agents such as diethyl cyanophosphate, diphenylphosphoryl azide and the like; carbonyldiimidazole, 2-chloro-1,3-dimethylimidazolium tetrafluoroborate and the like can be mentioned.

As the solvent to be used for the reaction using a condensing agent, for example, amides such as N,N-dimethylformamide, N,N-dimethylacetamide, 1-methyl-2-pyrrolidone and the like; halogenated hydrocarbons such as chloroform, dichloromethane and the like; aromatic hydrocarbons such as benzene, toluene and the like; ethers such as tetrahydrofuran, dioxane, diethyl ether and the like; esters such as ethyl acetate, propyl acetate, butyl acetate and the like; nitriles such as acetonitrile, propionitrile and the like; ketones such as acetone, 2-butanone and the like; water and the like can be mentioned. These solvents may be used in a combination of two or more kinds thereof at appropriate ratios.

The amount of the ammonia to be used is generally 1 to 10 molar equivalents, preferably 1 to 3 molar equivalents, relative to compound (V). The amount of the condensing agent to be used is generally 1 to 10 molar equivalents, preferably 1 to 3 molar equivalents, relative to compound (V).

When the aforementioned carbodiimide condensing agent is used as the condensing agent, the reaction efficiency can be improved by using a suitable condensation promoter (e.g., 1-hydroxy-7-azabenzotriazole, 1-hydroxybenzotriazole, N-hydroxysuccinimide, N-hydroxyphthalimide) as necessary. When the aforementioned phosphate condensing agent is used as the condensing agent, the reaction efficiency can be generally improved by adding an organic amine base such as triethylamine and the like.

As the ammonia to be used for this reaction, aqueous ammonia, alcoholic ammonia, ammonia-1-hydroxybenzotriazole complex, other salts with ammonia and the like can be mentioned, which is appropriately selected for the reaction.

The amount of the above-mentioned condensation promoters and ammonia to be used is generally 0.1 to 10 molar equivalents, preferably 0.3 to 3 molar equivalents, relative to compound (V).

The reaction temperature is generally −30° C. to 100° C.

The reaction time is generally 0.5 to 60 hrs.

In the method using the reactive derivative of compound (V), the reactive derivative is exemplified by acid anhydride, acid halide (e.g., acid chloride, acid bromide), imidazolide, mixed acid anhydride (e.g., anhydride with methyl carbonate, ethyl carbonate, isobutyl carbonate etc.) and the like.

For example, when acid halide is used as a reactive derivative of compound (V), for example, thionyl chloride, thionyl bromide, phosphorus trichloride, phosphorus tribromide, phosphorus oxychloride, phosphorus pentachloride and the like can be mentioned as the halogenating agent. The amount of the halogenating agent to be used is generally 0.1 to 10 molar equivalents, preferably 0.3 to 3 molar equivalents, relative to compound (V). In the reaction using the halogenating agent, amides such as N,N-dimethylformamide, N,N-dimethylacetamide and the like may be used as the catalyst. The amount of the catalyst to be used is generally 0.0001 to 10 molar equivalents, preferably 0.001 to 3 molar equivalents, relative to compound (V). In some cases, these catalysts may be used as a solvent.

As the solvent for acid halogenation, for example, amides such as N,N-dimethylformamide, N,N-dimethylacetamide, 1-methyl-2-pyrrolidone and the like; halogenated hydrocarbons such as chloroform, dichloromethane and the like; aromatic hydrocarbons such as benzene, toluene and the like; ethers such as tetrahydrofuran, dioxane, diethyl ether and the like; esters such as ethyl acetate, propyl acetate, butyl acetate and the like; nitriles such as acetonitrile, propionitrile and the like; ketones such as acetone, 2-butanone and the like; and the like can be mentioned. These solvents may be used in a combination of two or more kinds thereof at appropriate ratios.

The reaction temperature of the halogenation is generally −30° C. to 150° C.

The reaction time is generally 0.5 to 20 hrs.

The acid chloride and acid bromide produced as above are reacted with ammonia. The amidation reaction is carried out in the presence of a base in a solvent that does not adversely influence the reaction.

As the base, for example, amines such as triethylamine, N-ethyldiisopropylamine, N-methylmorpholine, N,N-dimethylaniline and the like; alkali metal salts such as sodium hydrogen carbonate, sodium carbonate, potassium carbonate and the like, and the like can be mentioned.

As the solvent that does not adversely influence the reaction, for example, halogenated hydrocarbons such as chloroform, dichloromethane and the like; aromatic hydrocarbons such as benzene, toluene and the like; ethers such as tetrahydrofuran, dioxane, diethyl ether and the like; esters such as methyl acetate, ethyl acetate, propyl acetate, butyl acetate and the like; nitriles such as acetonitrile, propionitrile and the like; amides such as N,N-dimethylformamide, N,N-dimethylacetamide, 1-methyl-2-pyrrolidone and the like; sulfoxides such as dimethyl sulfoxide and the like; water and the like can be mentioned. These solvents may be used in a combination of two or more kinds thereof at appropriate ratios.

The amount of the base to be used is generally 1 to 10 molar equivalents, preferably 1 to 3 molar equivalents, relative to a reactive derivative of compound (V). The reaction temperature is generally −30° C. to 150° C. The reaction time is generally 0.5 to 20 hrs.

When a mixed acid anhydride is used as the reactive derivative of compound (V), the reaction is carried out by, for example, reacting compound (V) with chlorocarbonate (e.g., methyl chlorocarbonate, ethyl chlorocarbonate, isobutyl chlorocarbonate) in the presence of a base and then reacting with ammonia.

As the base, for example, amines such as triethylamine, N-methylmorpholine, N-ethyldiisopropylamine, N,N-dimethylaniline and the like; alkali metal salts such as sodium hydrogen carbonate, sodium carbonate, potassium carbonate and the like; and the like can be mentioned.

The amount of the ammonia to be used is generally 1 to 10 molar equivalents, preferably 1 to 3 molar equivalents, relative to compound (V).

The reaction temperature is generally −30° C. to 100° C.

The reaction time is generally 0.5 to 20 hrs.

The thus-obtained compound (I) can be isolated and purified by known separation and purification means, such as concentration, concentration under reduced pressure, solvent extraction, crystallization, recrystallization, phase transfer, chromatography and the like.

The optically active compound (I) can be produced by the method described in the following. That is,

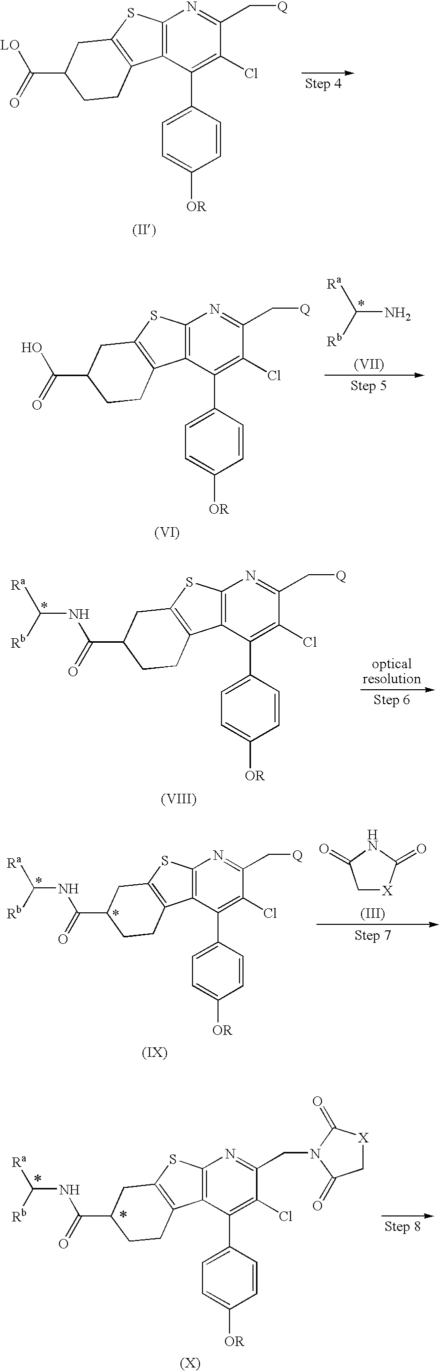

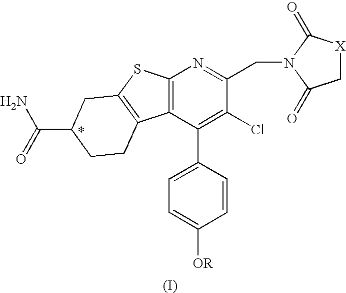

wherein the symbols in the formulas are as defined above.

### Step 4

In this step, the ester moiety of compound (II′) is hydrolyzed to give compound (VI).

This reaction is generally carried out in the presence of an acid in a solvent that does not adversely influence the reaction.

As the acid, for example, mineral acids such as hydrochloric acid, hydrobromic acid, sulfuric acid, nitric acid, phosphoric acid and the like, organic acids such as acetic acid, propionic acid, trichloroacetic acid, trifluoroacetic acid and the like, sulfonic acids such as methanesulfonic acid, ethanesulfonic acid, trifluoromethanesulfonic acid, benzenesulfonic acid and the like, and the like can be mentioned.

The amount of the acid to be used is generally 0.1 to 200 molar equivalents, preferably 1 to 100 molar equivalents, relative to compound (II′). The acid may be used as a solvent, and two or more kinds thereof may be used in combination at appropriate ratios.

The reaction temperature is generally −10 to 200° C., preferably 0 to 150° C.

The reaction time is generally 0.5 to 20 hrs.

As the solvent that does not adversely influence the reaction, for example, alcohols such as methanol, ethanol, propanol, isopropanol, methoxyethanol and the like; ethers such as dioxane, dimethoxyethane and the like; ketones such as acetone, 2-butanone and the like; amides such as N,N-dimethylformamide, N,N-dimethylacetamide, 1-methyl-2-pyrrolidone and the like; sulfoxides such as dimethyl sulfoxide and the like; water and the like can be mentioned. These solvents may be used in a combination of two or more kinds thereof at appropriate ratios.

Alternatively, this reaction may be carried out in the presence of a base in a solvent that does not adversely influence the reaction.

As the base, for example, alkali metal salts such as potassium hydroxide, sodium hydroxide, sodium hydrogen carbonate, potassium carbonate and the like can be mentioned.

The amount of the base to be used is 0.1 to 100 molar equivalents, preferably 1 to 20 molar equivalents, relative to compound (II′).

As the solvent that does not adversely influence the reaction, for example, aromatic hydrocarbons such as benzene, toluene, xylene and the like; ethers such as tetrahydrofuran, dioxane, diethyl ether and the like; halogenated hydrocarbons such as chloroform, dichloromethane and the like; ketones such as acetone, 2-butanone and the like; amides such as N,N-dimethylformamide, N,N-dimethylacetamide, 1-methyl-2-pyrrolidone and the like; nitrites such as acetonitrile, propionitrile and the like; sulfoxides such as dimethyl sulfoxide and the like; ater and the like can be mentioned. These solvents may be used in a combination of two or more kinds thereof at appropriate ratios.

The reaction temperature is generally −10 to 150° C., preferably 0 to 110° C.

The reaction time is generally 0.5 to 20 hrs.

### Step 5

In this step, compound (VI) reacted with optically active amine compound (VII) to give diastereomer compound (VIII).

As the optically active amine compound (VII), for example, (S)-(−)-α-methylbenzylamine, (R)-(+)-α-methylbenzylamine, (S)-α-methyl-4-nitrobenzylamine, (R)-α-methyl-4-nitrobenzylamine, (S)-(−)-1-(1-naphthyl)ethylamine, (R)-(+)-1-(1-naphthyl)ethylamine and the like can be mentioned.

This reaction is carried out by, for example, a method comprising directly condensing compound (VI) with compound (VII) sing a condensing agent, or a method comprising appropriately reacting a reactive derivative of compound (VI) with compound (VII) and the like.

As the condensing agent, for example, carbodiimide condensing agents such as dicyclohexylcarbodiimide, disopropylcarbodiimide, 1-ethyl-3-dimethylaminopropylcarbodiimide, a hydrochloride thereof and the like; phosphate condensing agents such as diethyl cyanophosphate, diphenylphosphoryl azide and the like; carbonyldiimidazole, 2-chloro-1,3-dimethylimidazolium tetrafluoroborate and the like can be mentioned.

As the solvent to be used for the reaction using a condensing agent, for example, amides such as N,N-dimethylformamide, N,N-dimethylacetamide, 1-methyl-2-pyrrolidone and the like; halogenated hydrocarbons such as chloroform, dichloromethane and the like; aromatic hydrocarbons such as benzene, toluene and the like; ethers such as tetrahydrofuran, dioxane, diethyl ether and the like; esters such as ethyl acetate, propyl acetate, butyl acetate and the like; nitrites such as acetonitrile, propionitrile and the like; ketones such as acetone, 2-butanone and the like; water and the like can be mentioned. These solvents may be used in a combination of two or more kinds thereof at appropriate ratios.

The amount of compound (VII) to be used is generally 1 to 10 molar equivalents, preferably 1 to 3 molar equivalents, relative to compound (VI).

The amount of the condensing agent to be used is generally 1 to 10 molar equivalents, preferably 1 to 3 molar equivalents, relative to compound (VI).

When the aforementioned carbodiimide condensing agent is used as the condensing agent, the reaction efficiency can be improved by using a suitable condensation promoter (e.g., 1-hydroxy-7-azabenzotriazole, 1-hydroxybenzotriazole, N-hydroxysuccinimide, N-hydroxyphthalimide) as necessary. In addition, when the aforementioned phosphate condensing agent is used as the condensing agent, the reaction efficiency can be generally improved by adding an organic amine base such as triethylamine and the like.

The amount of the above-mentioned condensation promoter and organic amine base to be used is generally 0.1 to 10 molar equivalents, preferably 0.3 to 3 molar equivalents, relative to compound (VI).

The reaction temperature is generally −30° C. to 100° C.

The reaction time is generally 0.5 to 60 hrs.

In a method using a reactive derivative of compound (VI), as the reactive derivative, for example, acid anhydride, acid halide (e.g., acid chloride, acid bromide), imidazolide, mixed acid anhydride (e.g., anhydride with methyl carbonate, ethyl carbonate, isobutyl carbonate) and the like can be mentioned.

For example, when acid halide is used as a reactive derivative of compound (VI), as the halogenating agent, for example, thionyl chloride, thionyl bromide, phosphorus trichloride, phosphorus tribromide, phosphorus oxychloride, phosphorus pentachloride and the like can be mentioned. The amount of the halogenating agent to be used is generally 0.1 to 10 molar equivalents, preferably 0.3 to 3 molar equivalents, relative to compound (VI). In the reaction using the halogenating agent, amides such as N,N-dimethylformamide, N,N-dimethylacetamide and the like may be used as the catalyst. The amount of the catalyst to be used is generally 0.0001 to 10 molar equivalents, preferably 0.001 to 3 molar equivalents, relative to compound (VI). In some cases, these catalysts can be used as the solvent.

As the solvent of the acid halogenation, for example, amides such as N,N-dimethylformamide, N,N-dimethylacetamide, 1-methyl-2-pyrrolidone and the like; halogenated hydrocarbons such as chloroform, dichloromethane and the like; aromatic hydrocarbons such as benzene, toluene and the like; ethers such as tetrahydrofuran, dioxane, diethyl ether and the like; esters such as ethyl acetate, propyl acetate, butyl acetate and the like; nitriles such as acetonitrile, propionitrile and the like; ketones such as acetone, 2-butanone and the like; and the like can be mentioned. These solvents may be used in a combination of two or more kinds thereof at appropriate ratios.

The reaction temperature of the halogenation is generally −30° C. to 150° C.

The reaction time is generally 0.5 to 20 hrs.

The acid chloride and acid bromide thus produced are reacted with optically active amine compound (VII). The amidation reaction is carried out in the presence of a base in a solvent that does not adversely influence the reaction.

As the base, for example, amines such as triethylamine, N-ethyldiisopropylamine, N-methylmorpholine, N,N-dimethylaniline and the like; alkali metal salts such as sodium hydrogen carbonate, sodium carbonate, potassium carbonate and the like; and the like can be mentioned.

As the solvent that does not adversely influence the reaction, for example, halogenated hydrocarbons such as chloroform, dichloromethane and the like; aromatic hydrocarbons such as benzene, toluene and the like; ethers such as tetrahydrofuran, dioxane, diethyl ether and the like; esters such as methyl acetate, ethyl acetate, propyl acetate, butyl acetate and the like; nitriles such as acetonitrile, propionitrile and the like; amides such as N,N-dimethylformamide, N,N-dimethylacetamide, 1-methyl-2-pyrrolidone and the like; sulfoxides such as dimethyl sulfoxide and the like; water and the like can be mentioned. These solvents may be used in a combination of two or more kinds thereof at appropriate ratios.

The amount of the reactive derivative of compound (VI) to be used is generally 1 to 10 molar equivalents, preferably 1 to 3 molar equivalents, relative to compound (VII).

The reaction temperature is generally −30° C. to 150° C.

The reaction time is generally 0.5 to 20 hrs.

When mixed acid anhydride is used as a reactive derivative of compound (VI), the reaction is carried out by, for example, reacting compound (VI) with chlorocarbonate (e.g., methyl chlorocarbonate, ethyl chlorocarbonate, isobutyl chlorocarbonate) in the presence of a base, and then reacting with compound (VII).

As the base, for example, amines such as triethylamine, N-methylmorpholine, N-ethyldiisopropylamine, N,N-dimethylaniline and the like; alkali metal salts such as sodium hydrogen carbonate, sodium carbonate, potassium carbonate and the like; and the like can be mentioned.

The amount of compound (VII) to be used is generally 1 to 10 molar equivalents, preferably 1 to 3 molar equivalents, relative to compound (VI).

The reaction temperature is generally −30° C. to 100° C.

The reaction time is generally 0.5 to 20 hrs.

The thus-obtained compound (VIII) can be isolated and purified by known separation and purification means, such as concentration, concentration under reduced pressure, solvent extraction, crystallization, recrystallization, phase transfer, chromatography and the like.

### Step 6

In this step, compound (VIII) is converted to optically active diastereomer compound (IX) by crystallization, recrystallization, chromatography and the like.

### Step 7

In this step, compound (IX) is reacted with compound (III) to give optically active compound (X).

This reaction is carried out according to a conventional method in the presence of a base in a solvent that does not adversely influence the reaction.

As the base, for example, alkali metal salts such as potassium hydroxide, sodium hydroxide, sodium hydrogen carbonate, potassium carbonate and the like; amines such as pyridine, triethylamine, N,N-dimethylaniline, 1,8-diazabicyclo[5.4.0]undeca-7-ene and the like; metal hydrides such as potassium hydride, sodium hydride and the like; alkali metal alkoxides such as sodium methoxide, sodium ethoxide, potassium t.-butoxide, sodium t.-butoxide and the like; and the like can be mentioned.

The amount of the base to be used is preferably 1 to 5 molar equivalents relative to compound (IX).

As the solvent that does not adversely influence the reaction, for example, aromatic hydrocarbons such as benzene, toluene, xylene and the like; ethers such as tetrahydrofuran, dioxane, diethyl ether and the like; ketones such as acetone, 2-butanone and the like; halogenated hydrocarbons such as chloroform, dichloromethane and the like; amides such as N,N-dimethylformamide, N,N-dimethylacetamide, 1-methyl-2-pyrrolidone and the like; nitrites such as acetonitrile, propionitrile and the like; sulfoxides such as dimethyl sulfoxide and the like; and the like can be mentioned. These solvents may be used in a combination of two or more kinds thereof at appropriate ratios.

The reaction temperature is generally −50 to 150° C., preferably −10 to 100° C.

The reaction time is generally 0.5 to 20 hrs.

Of the optically active compounds (X) obtained by this step, a compound represented by the formula

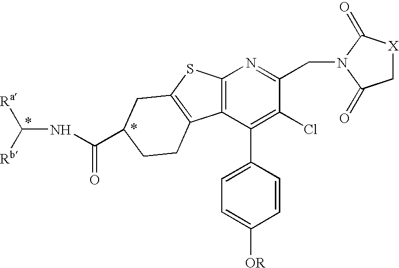

wherein the symbols are as defined above, and a salt thereof are novel compounds.

### Step 8

In this step, the amino-protecting group bonded to the amide nitrogen atom of the optically active compound (X), namely, a group represented by

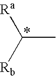

wherein R^{a}, R^{b} and * are as defined above is cleaved to give compound (I).

This reaction can be carried out in the presence of an acid. As the acid, for example, mineral acids such as hydrochloric acid, hydrobromic acid, sulfuric acid, nitric acid, phosphoric acid and the like, organic acids such as acetic acid, propionic acid, trichloroacetic acid, trifluoroacetic acid and the like, sulfonic acids such as methanesulfonic acid, ethanesulfonic acid, trifluoromethanesulfonic acid, benzenesulfonic acid and the like, and the like can be mentioned. While the amount of the acid to be used is 1 to 100 molar equivalents relative to optically active compound (X), the acid may be used as a solvent.

This reaction can be also carried out in a solvent that does not adversely influence the reaction. As the solvent that does not adversely influence the reaction, for example, aromatic hydrocarbons such as benzene, toluene, xylene and the like; ethers such as tetrahydrofuran, dioxane, diethyl ether and the like; ketones such as acetone, 2-butanone and the like; halogenated hydrocarbons such as chloroform, dichloromethane and the like; amides such as N,N-dimethylformamide, N,N-dimethylacetamide, 1-methyl-2-pyrrolidone and the like; nitrites such as acetonitrile, propionitrile and the like; sulfoxides such as dimethyl sulfoxide and the like; and the like can be mentioned. These solvents may be used in a combination of two or more kinds thereof at appropriate ratios.

In this reaction, a scavenger may be used. As the scavenger, for example, amino acids (cysteine, methoinine and the like) can be mentioned besides phenols (e.g., phenol, cresol and the like), anisole, veratrol and the like.

The reaction temperature is generally -20 to 200° C., preferably −10 to 150° C.

The reaction time is generally 0.5 to 20 hrs.

In addition, amide compound (X) can be produced by the method shown below.

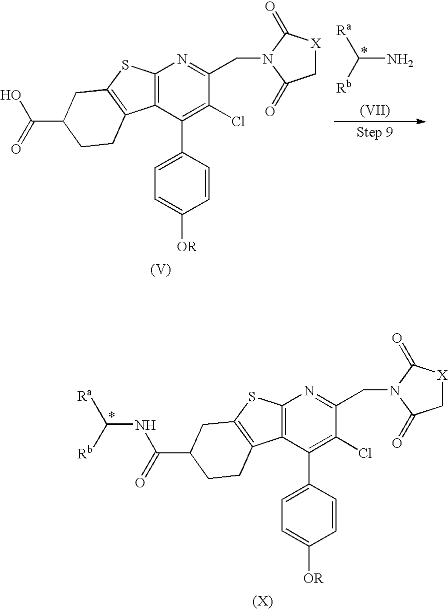

wherein each symbol is as defined above.

This reaction (step 9) can be carried out by the method mentioned in Step 5 or a method analogous thereto. The obtained compound (X) can be converted to optically active compound (I) by the aforementioned Step 6, the method mentioned in Step 8 or a method analogous thereto.

In addition, the optically active compound (I) can be also produced by the following method via the following ester compounds (XII) and (XIII).

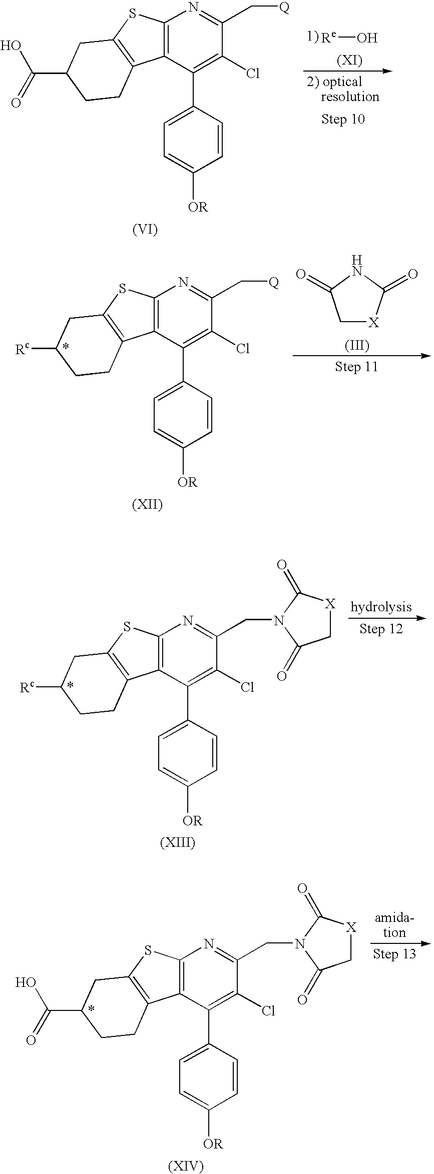

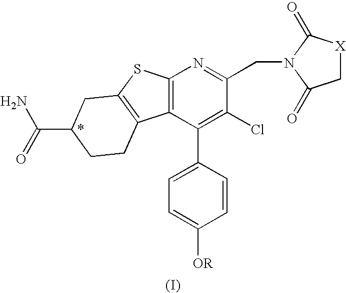

wherein the symbols in the formulas are as defined above.

### Step 10

In this step, compound (VI) is reacted with an alcohol compound having an optically active asymmetric carbon (XI) to give an ester compound, which is then optically resolved to give optically active diastereomer ester compound (XII). This reaction is carried out by the method mentioned in Step 5 or a method analogous thereto. The obtained ester compound is converted to optically diastereomer ester compound (XII) by means such as crystallization, recrystallization, chromatography and the like, in the same manner as in the aforementioned Step 6.

### Step 11

In this step, compound (XII) is reacted with compound (III) to give compound (XIII). This reaction is carried out by the method mentioned in Step 1 or a method analogous thereto.

### Step 12

In this step, the ester moiety of compound (XIII) is hydrolyzed to give compound (XIV). This reaction is carried out by the method mentioned in Step 2 or a method analogous thereto. Of the compounds (XIV), a compound represented by the formula

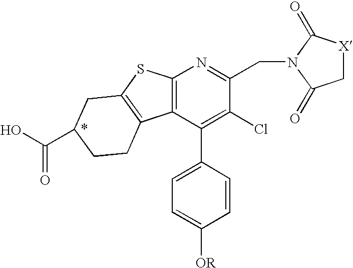

wherein the symbols are as defined above, and a salt thereof are novel compounds.

### Step 13

In this step, compound (XIV) is reacted with ammonia to give optically active compound (I). This reaction is carried out by the method mentioned in Step 3 or a method analogous thereto.

In each of the aforementioned reactions, when the starting compound has an amino group, a carboxyl group or a hydroxyl group as a substituent, these groups may be protected by a protecting group generally used in the peptide chemistry and the like. In this case, the object compound can be obtained by removing the protecting group after the reaction, as necessary. Introduction and removal of these protecting groups can be performed by a method known per se, such as the method described in Wiley-Interscience, 1999, “Protective Groups in Organic Synthesis, 3^{rd} Ed.” (authored by Theodora W. Greene, Peter G. M. Wuts), and the like.

The compound (I) can be also produced by further combining, where desired, one or more of known hydrolysis, deprotection, acylation reaction, alkylation reaction, oxidization reaction, ring-forming reaction, carbon chain extension reaction and substituent exchange reaction with the above-mentioned reaction.

The compound (I) can be isolated and purified by a known means, such as phase transfer, concentration, solvent extraction, fractionation, pH adjustment, crystallization, recrystallization, chromatography and the like.

When compound (I) is obtained as a free compound, it can be converted to an object salt by a method known per se or a method analogous thereto, and when compound (I) is obtained as a salt, it can be converted to a free form or an object other salt by a method known per se or a method analogous thereto.

As the salt of compound (I), pharmaceutically acceptable salts are preferable, such as metal salt, ammonium salt, salts with organic base, salts with inorganic acid, salts with organic acid, salts with basic or acidic amino acid and the like can be mentioned. As preferable examples of metal salts, for example, alkali metal salts such as sodium salt, potassium salt and the like; alkaline earth metal salts such as calcium salt, magnesium salt, barium salt and the like; aluminum salt and the like can be mentioned. As preferable examples of the salts with organic base, for example, salts with trimethylamine, triethylamine, pyridine, picoline, 2,6-lutidine, ethanolamine, diethanolamine, triethanolamine, tromethamine [tris(hydroxymethyl)methylamine], t-butylamine, cyclohexylamine, dicyclohexylamine, N,N′-dibenzylethylenediamine and the like can be mentioned. As preferable examples of salts with inorganic acid, for example, salts with hydrochloric acid, hydrobromic acid, nitric acid, sulfuric acid, phosphoric acid and the like can be mentioned. As preferable examples of salts with organic acid, for example, salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like can be mentioned. As preferable examples of salts with basic amino acid, for example, salts with arginine, lysin, ornithine and the like can be mentioned, and as preferable examples of acidic amino acid, for example, salts with aspartic acid, glutamic acid and the like can be mentioned.

Compound (I) may be used in the form of a prodrug. A prodrug of compound (I) is a compound that converts to compound (I) due to the reaction of enzyme, gastric acid and the like under the physiological conditions in the body. That is, a compound that converts to compound (I) by enzymatic oxidation, reduction, hydrolysis and the like, and a compound that converts to compound (I) by hydrolysis and the like by gastric acid and the like.

A prodrug of compound (I) is exemplified by a compound wherein an amino group of compound (I) is acylated, alkylated, phosphorylated (e.g., compound where amino group of compound (I) is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated, t-butylated and the like); compound wherein a hydroxy group of compound (I) is acylated, alkylated, phosphorinated, borated (e.g., compound where hydroxy group of compound (I) is acetylated, palmitoylated, propanoylated, pivaloylated, succinylated, fumarinated, alanylated, dimethylaminomethylcarbonylated and the like); compound wherein a carboxyl group of compound (I) is esterified or amidated (e.g., compound where carboxyl group of compound (I) is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified, methylamidated and the like) and the like. These compounds can be produced from compound (I) by a method known per se.

A prodrug of compound (I) may be a compound that converts to compound (I) under physiological conditions as described in Development of pharmaceutical products, vol. 7, Molecule Design, 163-198, Hirokawa Shoten (1990).

When compound (I) has an isomer such as optical isomer, stereo isomer, positional isomer, rotation isomer and the like, either one of the isomers and a mixture thereof are also encompassed in compound (I). For example, when compound (I) has an optical isomer, an optical isomer separated from the racemate is also encompassed in compound (I). These isomers can be obtained as a single product by a synthetic method, a separation method (concentration, solvent extraction, column chromatography, recrystallization and the like) and the like, which are known per se.

The compound (I) may be a crystal, and both a single crystal form and a mixture of crystal forms are encompassed in compound (I). The crystal can be produced by crystallization by a crystallization method known per se.

The compound (I) may be a solvate (e.g., hydrate etc.) or a non-solvate, and both are encompassed in compound (I).

The compounds labeled with an isotope (e.g., ³H, ¹⁴C, ³⁵S, ¹²⁵I and the like) and the like are encompassed in compound (I).

Since the compound (I) of the present invention or a prodrug thereof (hereinafter sometimes to be abbreviated as the compound of the present invention) shows an anti-inflammatory effect and further an antarthritic activity, it can be used for the prophylaxis or treatment of all conditions of arthritis (e.g., rheumatoid arthritis) showing inflammation symptoms in the joint.

The compound of the present invention has a superior bone resorption-suppressing activity, and is useful for the prophylaxis or treatment of bone destruction, osteoporosis and the like associated with arthritis.

Moreover, the compound of the present invention has an immune cytokine [e.g., interleukin-2 (IL-2), interferon-γ (IFN-γ) and the like] production-suppressing activity and is also useful for the prophylaxis or treatment of diseases considered to involve immunity including autoimmune diseases. As the target diseases, for example, systemic lupus erythematosus, inflammatory bowel diseases (ulcerative colitis, Crohn's disease), multiple sclerosis, psoriasis, chronic hepatitis, bladder cancer, breast cancer, carcinoma of uterine cervix, chronic lymphatic leukocyte, chronic bone marrow leukemia, bowel cancer, colon cancer, rectal cancer, Helicobacter pylori infectious disease, Hodgkin's disease, insulin dependent diabetes, malignant melanoma, multiple myeloma, non-Hodgkin's lymphoma, non-small cell lung cancer, ovarian cancer, digestive ulcer, prostate cancer, sepsis shock, tuberculosis, infertility, arteriosclerosis, Behcet's disease, asthma, atopic dermatitis, nephritis, systemic Fungi infectious disease, acute bacteria meningitis, acute cardiac infarction, acute pancreatitis, acute virus cerebritis, adult respiratory distress syndrome, bacteria pneumonia, chronic pancreatitis, herpes simplex virus infectious disease, chickenpox-herpes zoster virus infectious disease, AIDS, human papilloma virus infectious disease, influenza, invasive staphylococcus infectious disease, peripheral vessel disease, sepsis, interstitial liver disease, regional ileitis and the like can be mentioned. The compound of the present invention is used particularly for the prophylaxis or treatment of systemic lupus erythematosus, chronic hepatitis, interstitial liver disease, asthma, psoriasis, ulcerative colitis, Crohn's disease, regional ileitis or multiple sclerosis and the like.

In addition, the compound of the present invention is useful for the prophylaxis or treatment of rejection after organ transplantation.

The compound of the present invention is also useful as a T cell differentiation modulating agent. The T cell differentiation modulating agent is a generic term of compounds that modulate differentiation of T lymphocyte into type I T lymphocyte (T1 cell) or type II T lymphocyte (T2 cell). T1 cell is a T lymphocyte that mainly produces IFN-γ, IL-2 and TNFβ as cytokines, and includes CD4⁺T lymphocyte and CD8⁺T lymphocyte. T2 cell is a T lymphocyte that mainly produces IL-4, IL-5 and IL-10 as cytokines, and includes CD4⁺T lymphocyte and CD8⁺T lymphocyte. Therefore, T cell differentiation modulating agent can be used for the prophylaxis or treatment of arthritis and the above-mentioned various diseases.

Since the compound of the present invention shows low toxicity (e.g., acute toxicity, chronic toxicity, genetic toxicity, genital toxicity, cardiotoxicity, light toxicity, drug interaction, carcinogenicity and the like) and is superior in oral absorbability, and also in water-solubility, stability, pharmacokinetics (absorption property, distribution, metabolism, excretion and the like) and efficacy expression, it is useful as a pharmaceutical agent.

Therefore, the compound of the present invention can be used as an agent for the prophylaxis or treatment of inflammatory disease (e.g., arthritis, rheumatoid arthritis etc.) or autoimmune diseases, an agent for the prophylaxis or treatment of rejection after organ transplantation or as an agent for the prophylaxis or treatment of bone destruction, osteoporosis and the like associated with arthritis, in mammals inclusive of human (e.g., human, horse, bovine, swine, dog, cat, rat, mouse and the like).

As used herein, the above-mentioned “prophylaxis” of diseases means, for example, administration of a pharmaceutical agent containing the compound of the present invention to patients who have not yet developed the disease but predicted to have a high risk of onset due to a certain factor relating to the disease, or patients who have developed the disease but show no rational symptoms, or administration of a pharmaceutical agent containing the compound of the present invention to patients who, after treatment of the disease, concerned about the recurrence of the disease.

The compound of the present invention can be used in combination with, for example, (1) a cyclooxygenase suppressing agent (Cox-I, Cox-II suppressing agents), (2) disease-modifying antirheumatic drug and immune suppressing agent, (3) biological formulation, (4) analgesic and antiphlogistic, (5) therapeutic drug for bone diseases, (6) p38 MAP kinase inhibitor and/or TNF-α production inhibitor, (7) c-JUN N terminal kinase (JNK) inhibitor and the like.

(1) As the cyclooxygenase suppressing agents (Cox-I, Cox-II suppressing agent), for example, salicylic acid derivatives such as celecoxib, rofecoxib, aspirin and the like, diclofenac, indomethacin, loxoprofen and the like can be mentioned.
(2) As the disease-modifying antirheumatic drugs and immune suppressing agents, for example, methotrexate, leflunomide, Prograf, sulfasalazine, D-penicillamine, oral gold compounds and the like can be mentioned.
(3) As the biological formulations, for example, monoclonal antibodies (e.g., anti-TNF-α antibody, anti-IL-12 antibody, anti-IL-6 antibody, anti-ICAM-1 antibody, anti-CD4 antibody etc.), soluble receptors (e.g., soluble TNF-α receptor etc.) and protein ligands (IL-1 receptor antagonist etc.) can be mentioned.
(4) As the analgesics and antiphlogistics, for example, central nervous system analgesics (e.g., morphine, codeine, pentazocine etc.), steroids (e.g., prednisolone, dexamethasone, betamethasone etc.) and anti-inflammatory enzyme agents (e.g., bromelain, lysozyme, proctase etc.) can be mentioned.
(5) As the therapeutic drug for bone diseases (e.g., bone fracture, bone refracture, osteoporosis, osteohalisteresis, Paget's disease of bone, stiff myelitis, rheumatoid arthritis, osteoarthrosis of knee and destruction of joint tissues in diseases similar thereto etc.), for example, calcium formulation (e.g., calcium carbonate etc.), calcitonin formulation, vitamin D formulation (e.g., alfacalcidol etc.), sex hormones (e.g., estrogen, estradiol etc.), prostaglandin A₁, bisphosphonates, ipriflavones, fluorine compounds (e.g., sodium fluoride etc.), vitamin K₂, bone morphogenetic protein (BMP), fibloblast cell growth factor (FGF), platelet-derived growth factor (PDGF), transforming growth factor (TGF-β), insulin-like growth factors 1 and 2 (IGF-1, -2), parathyroid hormone (PTH) and the like can be mentioned.
(6) As the p38MAP kinase inhibitor and/or TNF-α production inhibitor, for example, the compounds described in WO 00/64894, WO 01/74811 and the like can be mentioned.
(7) As the JNK inhibitor, for example, the compounds described in WO 00/35906, WO 00/35909, WO 00/35921, WO 00/64872, WO 00/75118 and the like, and the like can be mentioned.

Examples of the administration mode of the compound of the present invention and a concomitant drug include the following: (1) The compound of the present invention and the concomitant drug are simultaneously produced to give a single preparation to be administered. (2) The compound of the present invention and the concomitant drug are separately produced to give two kinds of preparations which are administered simultaneously by the same administration route. (3) The compound of the present invention and the concomitant drug are separately produced to give two kinds of preparations which are administered by the same administration route only at the different times. (4) The compound of the present invention and the concomitant drug are separately produced to give two kinds of preparations which are administered simultaneously by the different administration routes. (5) The compound of the present invention and the concomitant drug are separately produced to give two kinds of preparations which are administered by the different administration routes only at different times (for example, the compound of the present invention and the concomitant drug are administered in this order, or in the reverse order) and the like. The dose of the concomitant drug can be appropriately determined based on the clinically employed dose. In addition, the mixing ratio of the compound of the present invention and the concomitant drug can be appropriately determined depending on the subject of administration, administration route, target disease, symptom, combination and the like. For example, when the subject of administration is a human, 0.01 to 100 parts by weight of a concomitant drug can be used relative to 1 part by weight of the compound of the present invention.

The compound of the present invention can be safely administered orally or parenterally (e.g., topical, rectal, intravenous injection etc.) as a single preparation or as a pharmaceutical composition, such as tablets (including sugar-coated tablet, film-coated tablet), powder, granule, capsule agent, liquid, emulsion, suspension, injection, suppository, sustained-release preparation, plaster and the like, which is obtained by admixing the compound with a pharmacologically acceptable carrier by a conventional method (e.g., the method described in the Japanese Pharmacopoeia etc.).

The content of the compound of the present invention in a pharmaceutical composition is about 0.01 to 100 wt % of the whole composition.

While the dose of the compound of the present invention can be variously determined according to the administration route and symptoms of patients to be treated, the amount of compound (I) can be selected from the ranges of, for example, about 1 mg to about 500 mg, preferably about 5 mg to about 100 mg, for oral administration to an adult, and about 0.1 mg to about 100 mg, more preferably about 0.3 mg to about 10 mg, for parenteral administration to an adult, which can be administered once to 3 portions divided for one day.

### EXAMPLES

The present invention is hereinafter described in more detail by means of the following Reference Examples, Examples, Formulation Examples and Experimental Examples, which are not to be construed as limitative, and may be changed without departing from the scope of the present invention.

The elution in column chromatography in Reference Examples and Examples was performed under the observation by a TLC (Thin Layer Chromatography), unless otherwise specified. TLC observation was performed using Silica Gel 60F₂₅₄ manufactured by Merck as a TLC plate and the solvent used as an elution solvent for column chromatography was used as a developing solvent. Detection was made by a UV detector. Silica gel 60 (70 to 230 mesh size) manufactured by Merck was employed as silica gel for column chromatography. A room temperature referred herein typically means a temperature from about 10° C. to 35° C. Moreover, sodium sulfate or magnesium sulfate was used for drying the extract.

Abbreviations employed in Reference Examples and Examples mean as follows.

LC: liquid chromatography

MS: mass analysis spectrum

ESI: electrospray ionization method

^{t}Bu: tert-butyl group, t-butyl group

Boc: tert-butyloxycarbonyl

rel: relative configuration

Rf: retardation factor

Rt: retention time

N: normal concentration

MPa: megapascal

wt %: weight percent

DMF: dimethylformamide

THF: tetrahydrofuran

DMSO: dimethyl sulfoxide

IPE: diisopropyl ether

CH₂Cl₂:methylene chloride

Et₂O: diethyl ether

HOBt.H₂O: 1-hydroxybenzotriazole hydrate

WSC: 1-ethyl-3-(dimethylaminopropyl)carbodiimide

WSC.HCl: 1-ethyl-3-(dimethylaminopropyl)carbodiimide hydrochloride

Et₃N: triethylamine

Boc₂O: di-tert-butyl dicarbonate

LC-MS in Examples and Reference Examples were measured under the following conditions.

### Analysis by LC-MS

Measurement instrument: LC-MS system (Waters Corporation)

HPLC portion: HP1100 (Agilent Technologies)

MS portion: ZMD (MicroMass)

### HPLC Conditions

Column: CAPCELL PAK C18 UG120, S-3 μm, 1.5×35 mm (Shiseido Co., Ltd.)

Solvents: solution A; 0.05% trifluoroacetic acid-containing water, solution B; 0.05% trifluoroacetic acid-containing acetonitrile

Gradient cycles: 0.00 min (solution A/solution B=90/10), 2.00 min (solution A/solution B=5/95), 2.75 min (solution A/solution B=5/95), 2.76 min (solution A/solution B=90/10), 3.60 min (solution A/solution B=90/10)

Injection volume: 2 μL, flow rate: 0.5 mL/min, detection method: UV 220 nm

### MS Conditions

Ionization method: ESI

### Analysis by LC

Measurement instrument: CLASS-VP system (Shimadzu Corporation)

### HPLC Conditions

Column: Inertsil ODS-2, CAPCELL PAK C18 UG120, 5 μm, 4.6×150 mm (GL Sciences Inc.)

Solvents: solution A; 0.1% trifluoroacetic acid-containing water, solution B; 0.1% trifluoroacetic acid-containing acetonitrile

Gradient cycles: 0.00 min (solution A/solution B=70/30), 15.00 min (solution A/solution B=15/85), 15.01 min (solution A/solution B=5/95), 20.00 min (solution A/solution B=5/95), 20.01 min (solution A/solution B=70/30), 25.00 min (solution A/solution B=70/30)

Injection volume: 10 μL, flow rate: 1.0 mL/min, detection method: UV 220 nm

The purification by preparative HPLC in Examples and Reference Examples was performed under the following conditions.

Instrument: high throughput purification system (Gilson)

Column: CombiPrep ODS-A S-5 μm, 50×20 mm (YMC)

Solvent: solution A; 0.1% trifluoroacetic acid-containing water, solution B; 0.1% trifluoroacetic acid-containing acetonitrile

Gradient cycle: 0.00 min (solution A/solution B=95/5), 1.00 min (solution A/solution B=95/5), 5.20 min (solution A/solution B=5/95), 6.40 min (solution A/solution B=5/95), 6.50 min (solution A/solution B=95/5), 6.60 min (solution A/solution B=95/5)

Flow rate: 25 mL/min, detection method: UV 220 nm Analysis by DSC

Measurement instrument: EXSTAR6000 DSC6200R (SII Nanotechnology)

### Measurement Conditions

Sample: 5 mg, measurement container: aluminum open pan, temperature rise rate: 5° C./min, measurement atmosphere: nitrogen gas 50 mL/min, measurement range: 25 to 300° C.

### Reference Example 1

### 4-Methoxybenzoylacetonitrile

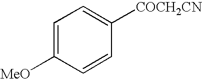

To a solution of methyl 4-methoxybenzoate (7.2 kg) in dimethyl sulfoxide (21.6 L) were added sodium methoxide (3.046 kg) and acetonitrile (2.135 kg), and the mixture was stirred at 110° C. for 2 hrs. Water (10.83 L) was added dropwise at not more than 15° C., and acetonitrile (14.4 L) was added. Then 6N HCl was added at not more than 20° C. to adjust its pH to 7.9, and the mixture was extracted with ethyl acetate (72 L). The aqueous layer was further extracted with ethyl acetate (36.32 L). The organic layers were combined and concentrated until the weight of the concentrate became 17.39 kg. Methanol (17.84 L) was added to the mixture, and water (17.84 L) was then added dropwise. The mixture was stirred at 5° C. for 1 hr, and the precipitated crystals were collected by filtration and washed with methanol-water (1:1) to give the title compound (6.40 kg, 82.7%).

¹H-NMR (CDCl₃) δ; 3.90 (3H, s), 4.03 (2H, s), 6.98 (2H, d, J=11.25 Hz), 7.90 (2H, s, J=11.25 Hz).

### Reference Example 2

### 2-Amino-4,5,6,7-tetrahydro-3-(4-methoxybenzoyl)-1-benzothiophene-6-carboxylic Acid Ethyl Ester

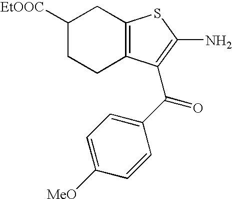

A mixture of 4-methoxybenzoylacetonitrile (13.6 g) obtained in Reference Example 1, cyclohexanone-4-carboxylic acid ethyl ester (14.0 g), sulfur (2.7 g), morpholine (7.3 g) and ethanol (300 ml) was stirred for 3 hrs with heating under reflux. After the completion of the reaction, the reaction solution was concentrated under reduced pressure. The obtained residue was purified by column chromatography (developing solvent: ethyl acetate-hexane (1:1, v/v)) to give the title compound as yellow crystals (25.0 g, 87%), which were recrystallized from ethyl acetate-hexane. Melting point: 102-103° C.

¹H-NMR (CDCl₃) δ; 1.26 (3H, t, J=7.0 Hz), 1.50-1.70 (1H, m), 1.87-2.18 (3H, m), 2.61-2.87 (3H, m), 3.86 (3H, s), 4.15 (2H, q, J=7.0 Hz), 6.30 (2H, brs), 6.90 (2H, d, J=8.8 Hz), 7.51 (2H, d, J=8.8 Hz).

### Reference Example 3

### 3-Chloro-2-chloromethyl-5,6,7,8-tetrahydro-4-(4-methoxyphenyl)[1]benzothieno[2,3-b]pyridine-7-carboxylic Acid Ethyl Ester

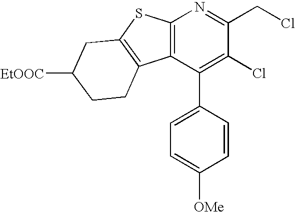

To a solution of the compound (10.0 g) obtained in Reference Example 2 and 1,3-dichloroacetone (6.0 g) in absolute THF (200 ml) was added aluminum chloride (7.4 g) under ice-cooling. The mixture was stirred at the same temperature for 5 min. and further 1.5 hrs with heating under reflux. The reaction mixture was cooled, and added to a mixed solution of toluene (400 ml) and ice water (100 ml) with stirring, and the mixture was further stirred at room temperature for 30 min. The organic layer was separated, washed with water and saturated brine and dried over magnesium sulfate. The solvent was removed by evaporation under reduced pressure to give the title compound as colorless crystals (8.5 g, 68%). The crystals were recrystallized from ethyl acetate-hexane. Melting point: 113-114° C.

¹H-NMR (CDCl₃) δ; 1.24 (3H, t, J=7.4 Hz), 1.58-1.80 (1H, m), 1.81-2.10 (3H, m), 2.61-2.81 (1H, m), 3.04-3.19 (2H, m), 3.90 (3H, s), 4.15 (2H, q, J=7.4 Hz), 4.94 (2H, s), 6.95-7.04 (2H, m), 7.06-7.22 (2H, m).

### Reference Example 4

### 3-Chloro-2-[(2,4-dioxo-1,3-oxazolidin-3-yl)methyl]-5,6,7,8-tetrahydro-4-(4-methoxyphenyl)[1]-benzothieno[2,3-b]pyridine-7-carboxylic Acid Ethyl Ester

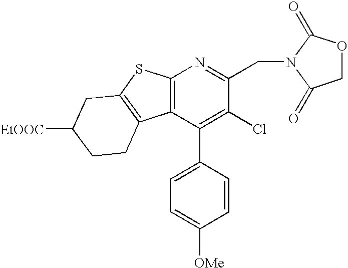

A mixture of the compound (2.4 g) obtained in Reference Example 3, oxazolidine-2,4-dione (808 mg), potassium carbonate (1.1 g) and N,N-dimethylformamide (DMF, 40 ml) was stirred under heating at 80° C. for 2 hrs. The reaction mixture was poured into water (100 ml), and extracted with ethyl acetate. The extract layer of ethyl acetate was washed with water and saturated brine and dried over magnesium sulfate. The solvent was removed by evaporation under reduced pressure to give the title compound as colorless crystals (2.2 g, 81%), which were recrystallized from ethyl acetate-hexane. Melting point: 118-119° C.

¹H-NMR (CDCl₃) δ; 1.24 (3H, t, J=7.4 Hz), 1.58-1.77 (1H, m), 1.84-2.07 (3H, m), 2.60-2.80 (1H, m), 3.00-3.10 (2H, m), 3.90 (3H, s), 4.14 (2H, q, J=7.4 Hz), 4.93 (2H, s), 5.06 (2H, s), 6.95-7.06 (2H, m), 7.07-7.23 (2H, m).

Furthermore, the compounds as shown in Reference Examples 5 and 6 were synthesized according to a method similar to that of Reference Example 4.

### Reference Example 5

### 3-Chloro-2-[(2,5-dioxo-1-pyrrolidinyl)methyl]-5,6,7,8-tetrahydro-4-(4-methoxyphenyl)[1]benzothieno[2,3-b]pyridine-7-carboxylic Acid Ethyl Ester

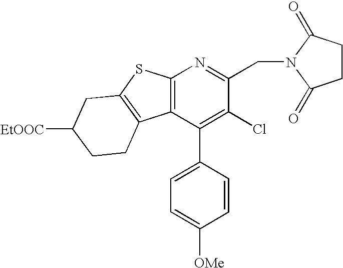

Melting point: 180-181° C. (solvent for recrystallization: ethyl acetate-hexane).

### Reference Example 6

### 3-Chloro-2-[(2,4-dioxo-1,3-thiazolidin-3-yl)methyl]-5,6,7,8-tetrahydro-4-(4-methoxyphenyl)[1]benzothieno[2,3-b]pyridine-7-carboxylic Acid Ethyl Ester

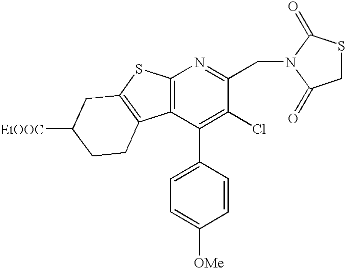

Melting point: 195-196° C. (solvent for recrystallization: ethyl acetate-hexane).

### Reference Example 7

### 3-Chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxylic Acid

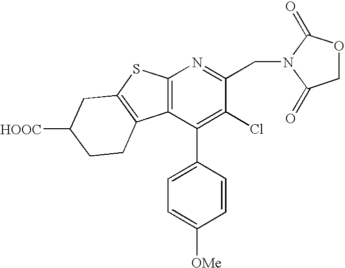

A mixed solution of the compound (2.0 g) obtained in Reference Example 4, 10% hydrochloric acid (20 ml) and dioxane (20 ml) was stirred with heating at 80° C. for 3.5 hrs. The reaction solution was poured into water (200 ml) and extracted with ethyl acetate. The organic layer was washed with water and saturated brine and dried over magnesium sulfate. The solvent was removed by evaporation under reduced pressure to give the title compound as colorless crystals (1.6 g, 84%), which were recrystallized from THF-hexane. Melting point: 218-219° C.

¹H-NMR (CDCl₃) δ; 1.60-2.10 (4H, m), 2.68-2.83 (1H, m), 3.00-3.18 (2H, m), 3.90 (3H, s), 4.93 (2H, s), 5.05 (2H, s), 6.95-7.23 (4H, m).

Furthermore, the compounds as shown in Reference Examples 8 and 9 were synthesized according to a method similar to that of Reference Example 7.

### Reference Example 8

### 3-Chloro-2-[(2,5-dioxo-1-pyrrolidinyl)methyl]-5,6,7,8-tetrahydro-4-(4-methoxyphenyl)[1]benzothieno[2,3-b]pyridine-7-carboxylic Acid

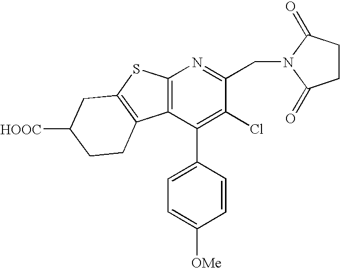

Melting point: 258-259° C. (solvent for recrystallization: THF-hexane).

### Reference Example 9

### 3-Chloro-2-[(2,4-dioxo-1,3-thiazolidin-3-yl)methyl]-5,6,7,8-tetrahydro-4-(4-methoxyphenyl)[l]benzothieno[2,3-b]pyridine-7-carboxylic Acid

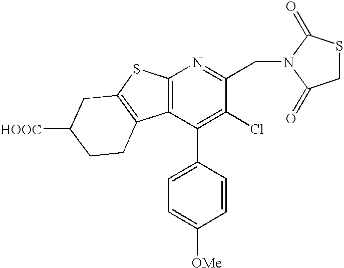

Melting point: 246-247° C. (solvent for recrystallization: THF-hexane).

### Reference Example 10

### 3-Chloro-2-chloromethyl-5,6,7,8-tetrahydro-4-(4-methoxyphenyl)[1]benzothieno[2,3-b]pyridine-7-carboxylic acid

3-Chloro-2-chloromethyl-5,6,7,8-tetrahydro-4-(4-methoxyphenyl)[1]benzothieno[2,3-b]pyridine-7-carboxylic acid ethyl ester (9.01 g) obtained in Reference Example 3, acetic acid (54 ml) and 4N HCl (27 ml) were mixed, and the mixture was stirred at 92° C. for 5 hrs. Water (54 ml) was added dropwise at 80° C. and the mixture was cooled. The precipitated crystals were collected by filtration, and washed with cool water (20 ml×3) to give the title compound as white crystals (8.27 g, yield 97.9%).

¹H-NMR (300 MHz, DMSO-d₆) δ; 1.57-1.63 (1H, m), 1.78-1.97 (3H, m) 2.71-2.76 (1H, m), 3.00-3.09 (2H, m), 3.92 (3H, s), 4.98 (2H, s), 7.06-7.28 (4H, m).

### Reference Example 11

### 3-Chloro-2-chloromethyl-5,6,7,8-tetrahydro-4-(4-methoxyphenyl)-N—[(1R)-1-phenylethyl][1]benzothieno[2,3-b]pyridine-7-carboxamide

3-Chloro-2-chloromethyl-5,6,7,8-tetrahydro-4-(4-methoxyphenyl)[1]benzothieno[2,3-b]pyridine-7-carboxylic acid (5 g) obtained in Reference Example 10 was dissolved in tetrahydrofuran (50 ml). DMF (0.05 g) was added and thionyl chloride (1.04 ml) was then added dropwise, and the mixture was stirred at 60° C. for 1 hr. The reaction mixture was concentrated under reduced pressure, and the residue was dissolved in N,N-dimethylacetamide (25 ml) and cooled. (R)-(+)-1-Phenylethylamine (1.81 ml) was added dropwise, and the mixture was stirred at room temperature for 3 hrs. To the reaction mixture was added acetonitrile (50 ml), and water (25 ml) was added dropwise. The precipitated crystals were collected by filtration and washed with acetonitrile-water (2:1, 25 ml×2) to give the title compound (5.13 g, yield 82.5%).

¹H-NMR (300 MHz, CDCl₃) δ; 1.48 (3H, m), 1.67-1.85 (5H, m), 2.44-2.51 (1H, m), 2.93-3.16 (2H, m), 3.88 (3H, s), 4.93 (2H, s), 5.07-5.17 (1H, m), 5.73-5.75 (1H, m), 6.95-7.32 (9H, m).

### Reference Example 12

### (7S)-3-Chloro-2-chloromethyl-5,6,7,8-tetrahydro-4-(4-methoxyphenyl)-N-[(1R)-1-phenylethyl][1]benzothieno[2,3-b]pyridine-7-carboxamide

3-Chloro-2-chloromethyl-5,6,7,8-tetrahydro-4-(4-methoxyphenyl)-N-[(1R)-1-phenylethyl][1]benzothieno[2,3-b]pyridine-7-carboxamide (5 g) obtained in Reference Example 11 was dissolved in methyl ethyl ketone (100 ml) with heating. The temperature was allowed to return to room temperature, and the mixture was stirred at the same temperature. The precipitated crystals were collected by filtration and washed with methyl ethyl ketone (10 ml) to give the title compound as white crystals (1.82 g, yield 29.3%).

¹H-NMR (300 MHz, CDCl₃) δ; 1.48 (3H, m), 1.67-1.85 (5H, m), 2.44-2.51 (1H, m), 2.93-3.16 (2H, m), 3.88 (3H, s), 4.93 (2H, s), 5.07-5.17 (1H, m), 5.73-5.75 (1H, m), 6.95-7.32 (9H, m).

### Reference Example 13

### (7S)-3-Chloro-2-[(2,4-dioxo-1,3-oxazolidin-3-yl)methyl]-5,6,7,8-tetrahydro-4-(4-methoxyphenyl)-N-[(1R)-1-phenylethyl][1]benzothieno[2,3-b]pyridine-7-carboxamide

(7S)-3-Chloro-2-chloromethyl-5,6,7,8-tetrahydro-4-(4-methoxyphenyl)-N-[(1R)-1-phenylethyl][1]benzothieno[2,3-b]pyridine-7-carboxamide (1.2 g) obtained in Reference Example 12, 2,4-dioxo-1,3-oxazolidine (0.25 g) and potassium carbonate (0.47 g) were added to N,N-dimethylformamide (6 ml), and the mixture was stirred at 80° C. for 1 hr. The mixture was allowed to cool to room temperature, and acetonitrile (6 ml), 2N HCl (2 ml) and water (7 ml) were added dropwise. The precipitated crystals were collected by filtration and washed with acetonitrile-water (1:1, 6 ml) to give the title compound as white crystals (1.27 g, yield 94.3%).

¹H-NMR (300 MHz, CDCl₃) δ; 1.48 (3H, m), 1.67-1.88 (5H, m), 2.41-2.50 (1H, m), 2.88-3.17 (2H, m), 3.88 (3H, s), 4.91 (2H, s), 4.99 (2H, s), 5.05-5.17 (1H, m), 5.71-5.73 (1H, m), 6.94-7.34 (9H, m).

### Reference Example 14

### (7S)-3-Chloro-2-[(2,5-dioxo-1-pyrrolidinyl)methyl]-5,6,7,8-tetrahydro-4-(4-methoxyphenyl)-N-[(1R)-1-phenylethyl][1]benzothieno[2,3-b]pyridine-7-carboxamide

(7S)-3-Chloro-2-chloromethyl-5,6,7,8-tetrahydro-4-(4-methoxyphenyl)-N-[(1R)-1-phenylethyl][1]benzothieno[2,3-b]pyridine-7-carboxamide (3.0 g) obtained in Reference Example 12, succinimide (0.62 g) and potassium carbonate (1.18 g) were added to N,N-dimethylformamide (15 ml), and the mixture was stirred at 80° C. for 1 hr. The mixture was cooled to room temperature, and acetonitrile (22.5 ml), 1N HCl (10 ml) and water (12.5 ml) were added dropwise. The precipitated crystals were collected by filtration, and washed with acetonitrile-water (1:1, 15 ml×2) to give the title compound (3.08 g, yield 91.7%).

¹H-NMR (300 MHz, CDCl₃) δ; 1.47 (3H, d), 1.65-1.87 (4H, m), 2.41-2.49 (1H, m), 2.88-3.16 (7H, m), 3.88 (3H, s), 4.95 (2H, s), 5.07-5.17 (1H, m), 5.72-5.75 (1H, d), 6.94-7.31 (9H, m).

### Reference Example 15

### (7S)-3-Chloro-2-[(2,4-dioxo-1,3-thiazolidin-3-yl)methyl]-5,6,7,8-tetrahydro-4-(4-methoxyphenyl)-N-[(1R)-1-phenylethyl][1]benzothieno[2,3-b]pyridine-7-carboxamide

(7S)-3-Chloro-2-chloromethyl-5,6,7,8-tetrahydro-4-(4-methoxyphenyl)-N-[(1R)-1-phenylethyl][1]benzothieno[2,3-b]pyridine-7-carboxamide (3.0 g) obtained in Reference Example 12,2,4-dioxo-1,3-thiazolidine (0.74 g) and potassium carbonate (1.18 g) were added to N,N-dimethylformamide (15 ml), and the mixture was stirred at 80° C. for 1 hr. The mixture was cooled to room temperature, and acetonitrile (22.5 ml), 1N HCl (10 ml) and water (12.5 ml) were added dropwise. The precipitated crystals were collected by filtration and washed with acetonitrile-water (1:1, 15 ml×2) to give the title compound as pale-brown crystals (3.35 g, yield 97.1%).

¹H-NMR (300 MHz, CDCl₃) δ; 1.46-1.48 (3H, m), 1.59-1.87 (4H, m), 2.41-2.49 (1H, m), 2.87-3.16 (3H, m), 3.88 (3H, s), 4.13 (2H, s), 5.07-5.16 (3H, m), 5.76-5.78 (1H, d), 6.94-7.31 (9H, m).

### Reference Example 16

### 3-Chloro-2-[(2,4-dioxo-1,3-oxazolidin-3-yl)methyl]-5,6,7,8-tetrahydro-4-(4-methoxyphenyl)-N-[(1R)-1-phenylethyl][1]benzothieno[2,3-b]pyridine-7-carboxamide

3-Chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxylic acid (0.20 g) obtained in Reference Example 7 was suspended in toluene (5 ml), and thionyl chloride (0.039 ml) was added at room temperature. Dimethylformamide (1 drop) was added, and the mixture was stirred at 50° C. for 1 hr. The solvent was removed by evaporation under reduced pressure. Toluene (5 ml) was added to the residue and the solvent was evaporated again under reduced pressure. The residue was dissolved by adding dimethylacetamide (4 ml). (R)-(+)-1-Phenylethylamine (0.063 ml) was added under ice-cooling, and the mixture was stirred at the internal temperature of 50° C. for 1 hr. The mixture was cooled to room temperature, and acetonitrile (4 ml) was added. Pure water (4 ml) was then added dropwise at room temperature. The mixture was stirred at room temperature for 1 hr, and the precipitated crystals were collected by filtration and washed with acetonitrile-pure water (1:1, 1 ml) to give the title compound (171 mg, 70.5%).

¹H-NMR (300 MHz, CDCl₃) δ; 1.45-1.53 (3H, m), 1.66-1.72 (1H, m), 1.83-1.90 (3H, m), 2.40-2.50 (1H, m), 2.94-3.14 (2H, m), 3.89 (3H, s), 4.92 (1H, s), 5.05 (1H, s), 5.10-5.15 (1H, m), 5.67-5.70 (1H, m), 6.96-7.34 (9H, m).

### Reference Example 17

### (3R)-4,4-Dimethyl-2-oxotetrahydrofuran-3-yl (7S)-3-chloro-2-chloromethyl-5,6,7,8-tetrahydro-4-(4-methoxyphenyl)[1]benzothieno[2,3-b]pyridine-7-carboxylate

3-Chloro-2-chloromethyl-5,6,7,8-tetrahydro-4-(4-methoxyphenyl)[1]benzothieno[2,3-b]pyridine-7-carboxylic acid (3.00 g) obtained in Reference Example 10, (S)-(+)-pantolactone (0.92 g), 1-ethyl-3-(3-dimethylaminopropyl)carbodiimide hydrochloride (WSC, 1.63 g) and 4-dimethylaminopyridine (DMAP, 86.8 mg) were added to DMF (15 ml), and the mixture was stirred at room temperature for 2 hrs. Ethyl acetate (45 ml) and water (36 ml) were added to the mixture. The organic layer was separated and washed successively with 1N HCl (24 ml), 10% brine (24 ml), 7% aqueous sodium hydrogen carbonate solution (24 ml) and 10% brine (24 ml). The solvent was evaporated and ethyl acetate (9 ml) was added. The mixture was stirred at the internal temperature of 55 to 58° C., whereby crystals were precipitated. Methanol (27 ml) was then added dropwise over about 10 min., and the mixture was stirred at the same temperature for 30 min. and at room temperature for 1 hr. The precipitated crystals were collected by filtration and washed with ethyl acetate-methanol=1:3 (6 ml) to give the title compound. Yield: 1.58 g (41.62%). Optical purity was 51.98% de.

### Conditions for Measurement of Optical Purity

### Column: CHIRALCEL OJ-R

Mobile phase: 0.05 M KH₂PO₄: CH₃CN=1:1
Flow rate: 0.7 ml/min.

### Temperature: 25° C.

Measurement wavelength: 262 nm

The obtained crystals (1.58 g) were suspended in THF (7.9 ml) and dissolved at the internal temperature of 55 to 58° C. Methanol (15.8 ml) was added dropwise at the same temperature for about 10 min. After the completion of the dropwise addition, the mixture was stirred at the same temperature for 30 min, and further stirred at room temperature for 1 hr. The crystals were collected by filtration and washed with THF-methanol (1:2, 3.2 ml). Yield: 1.10 g (69.62%). Optical purity: 78.70% de.

The crystals (1.10 g) obtained as mentioned above were dissolved in THF (5.5 ml) at the internal temperature of 55 to 58° C. Methanol (11.0 ml) was added dropwise at the same temperature over about 10 min. After the completion of the dropwise addition, the mixture was stirred at the same temperature for 30 min, and further stirred at room temperature for 1 hr. The crystals were collected by filtration and washed with THF-methanol (1:2, 2.2 ml). Yield: 0.93 g (84.55%).

Optical purity: 96.96% de.

¹H-NMR (300 MHz, CDCl₃) δ; 1.09 (3H, s), 1.19 (3H, s), 1.73-1.78 (1H, m), 1.96-2.09 (3H, m), 2.92-2.99 (1H, m), 3.18 (2H, d, J=7.3 Hz), 3.90 (3H, s), 4.00-4.07 (2H, m), 4.94 (2H, s), 5.37 (1H, s), 6.99-7.02 (2H, m), 7.12-7.16 (1H, m), 7.19-7.22 (1H, m).

### Reference Example 18

### (7S)-3-Chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxylic acid

(3R)-4,4-Dimethyl-2-oxotetrahydrofuran-3-yl (7S)-3-chloro-2-chloromethyl-5,6,7,8-tetrahydro-4-(4-methoxyphenyl)[1]benzothieno[2,3-b]pyridine-7-carboxylate (1.22 g) obtained in Reference Example 17 was dissolved in acetic acid (5.1 ml). To the mixture was added 4N HCl (2.6 ml), and the mixture was stirred for 3 hrs at the internal temperature of 95 to 100° C. The mixture was cooled to the internal temperature of 50° C., and water (7.65 ml) was added dropwise. The mixture was stirred at room temperature for 1 hr, and the crystals were collected by filtration and washed with water to give the title compound. Yield: 0.66 g.

¹H-NMR (300 MHz, DMSO-d₆) δ; 1.53-1.60 (1H, m), 1.75-1.97 (3H, m), 2.66-2.73 (1H, m), 2.90-3.07 (2H, m), 3.83 (3H, s), 4.90 (2H, s), 5.12 (2H, s), 7.04-7.07 (2H, m), 7.19-7.27 (2H, m).

### Reference Example 19

### (7S)-3-Chloro-2-[(2,4-dioxo-1,3-oxazolidin-3-yl)methyl]-5,6,7,8-tetrahydro-4-(4-methoxyphenyl)-N-[(1R)-1-phenylethyl][1]benzothieno[2,3-b]pyridine-7-carboxamide

3-Chloro-2-[(2,4-dioxo-1,3-oxazolidin-3-yl)methyl]-5,6,7,8-tetrahydro-4-(4-methoxyphenyl)-N-[(1R)-1-phenylethyl][1]benzothieno[2,3-b]pyridine-7-carboxamide (5.00 g) obtained in Reference Example 16 was suspended in acetonitrile (100 ml), and the mixture was stirred with heating under reflux for 15 min. The mixture was gradually cooled to the internal temperature of 40° C. for about 1 hr, and stirred at the same temperature for 1 hr. The precipitated crystals were collected by filtration, and washed with acetonitrile (10 ml) warmed to 40° C. to give crystals (2.13 g, 42.6%). Optical, purity: 94.4% de.

The obtained crystals (2.00 g) were suspended in acetonitrile-pure water (9:1, 20 ml) and the suspension was heated under reflux for 1 hr (the crystals did not dissolve completely). Pure water (4 ml) was added dropwise at the internal temperature of not less than 70° C., and the mixture was gradually cooled to room temperature for about 0.5 hr. The mixture was stirred at room temperature for 1 hr. The precipitated crystals were collected by filtration and washed with acetonitrile-pure water (3:1, 2 ml) to give the title compound (1.9 g). Optical purity: 99.2% de.

¹H-NMR (300 MHz, CDCl₃) δ; 1.46-1.49 (3H, m), 1.60-1.70 (1, m), 1.80-1.90 (3H, m), 2.43-2.48 (1H, m), 2.94-3.14 (2H, m), 3.88 (3H, s), 4.91 (1H, s), 5.05 (1H, s), 5.07-5.15 (1H, s), 5.70-5.73 (1H, s), 6.95-7.34 (9H, s).

### Conditions for Measurement of Optical Purity

Detection wavelength: UV 262 nm

### Column: DAICEL CHIRALCEL OJ-R, 4.6 mm i.d. (150 mm

Column temperature: 25° C.
Mobile phase: 0.05 M KH₂PO₄: CH₃CN=1:1
Flow: 1.0 mL/min

### Reference Example 20

### (3R)-4,4-Dimethyl-2-oxotetrahydrofuran-3-yl (7S)-3-chloro-2-[(2,4-dioxo-1,3-oxazolidin-3-yl)methyl]-5,6,7,8-tetrahydro-4-(4-methoxyphenyl) [1]benzothieno[2,3-b]pyridine-7-carboxylate

(3R)-4,4-Dimethyl-2-oxotetrahydrofuran-3-yl (7S)-3-chloro-2-chloromethyl-5,6,7,8-tetrahydro-4-(4-methoxyphenyl) [1]benzothieno[2,3-b]pyridine-7-carboxylate (0.85 g) obtained in Reference Example 17, 2,4-dioxo-1,3-oxazolidine (176.8 mg) and potassium carbonate (263.8 mg) were suspended in DMF (4.25 ml), and the suspension was stirred at the internal temperature of 80 to 85° C. for 2 hrs. The suspension was cooled to room temperature. Ethyl acetate (17 ml) and water (8.5 ml) were added, and the organic layer was separated and washed with water and 10% brine. The solvent was removed by evaporation to give the title compound as a yellow oil (1.22 g). The product was used in the next step without purification.

¹H-NMR (300 MHz, CDCl₃) δ; 1.08 (3H, s), 1.18 (3H, s), 1.73-1.76 (1H, m), 1.93-2.07 (3H, m), 2.90-2.95 (1H, m), 3.14 (2H, d, J=7.3 Hz), 3.89 (3H, s), 4.03-4.06 (2H, m), 4.93 (2H, s), 5.05 (2H, s), 5.35 (1H, s), 6.98-7.01 (2H, m), 7.09-7.19 (2H, m).

### Example 1

### 3-Chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxamide

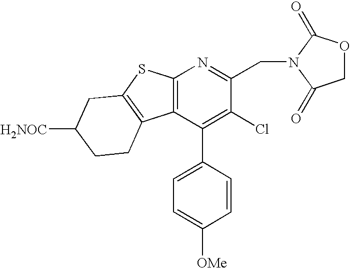

A mixed solution of the compound obtained in Reference Example 7 (3-chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxylic acid, 500 mg), NH₃* HOBt (172 mg), WSC (217 mg) and DMF (10 ml) was stirred at room temperature for 12 hrs. The reaction solution was poured into water (30 ml) and extracted with ethyl acetate. The extract layer of ethyl acetate was washed with water and saturated brine and dried over magnesium sulfate. The solvent was removed by evaporation under reduced pressure to give the title compounds as colorless crystals (2.1 g, 84%), which were recrystallized from ethyl acetate-hexane. Melting point: 155-156° C.

¹H-NMR (CDCl₃) δ; 1.58-2.10 (4H, m), 2.45-2.63 (1H, m), 2.90-3.15 (2H, m), 3.90 (3H, s), 4.93 (2H, s), 5.06 (2H, s), 5.39 (1H, brs), 5.46 (1H, brs), 6.96-7.30 (4H, m).

The compounds as shown in Examples 2 and 3 were synthesized according to a method similar to that of Example 1.

### Example 2

### 3-Chloro-2-[(2,5-dioxo-1-pyrrolidinyl)methyl]-5,6,7,8-tetrahydro-4-(4-methoxyphenyl))[1]benzothieno[2,3-b]pyridine-7-carboxamide

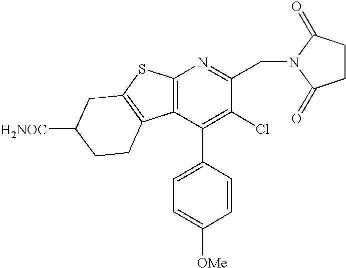

Melting point: 294-295° C. (solvent for recrystallization: ethyl acetate-hexane).

### Example 3

### 3-Chloro-2-[(2,4-dioxo-1,3-thiazolidin-3-yl)methyl]-5,6,7,8-tetrahydro-4-(4-methoxyphenyl)[1]benzothieno[2,3-b]pyridine-7-carboxamide

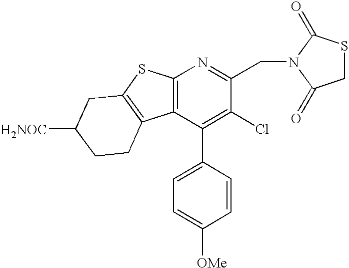

Melting point: 162-163° C. (solvent for recrystallization: acetone-hexane).

### Example 4

The compound obtained in Example 1 (3-chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxamide, 280 mg) was fractionated by high performance liquid chromatography (HPLC) (column: CHIRALPAK AD 50 mm ID×500 mm L (AD00CM-AK001), temperature 20° C., mobile phase: hexane/ethanol=85/15, flow rate: 80 ml/min, detection wavelength: 254 nm, 1 shot: 50 mg), and concentrated to dryness. The residue was dissolved in ethanol and passed through a filter (0.45 μm). The filtrate was concentrated, hexane was added, and the mixture was concentrated again to dryness. An enantiomer form (Example 4-1) in which retention time was short and the optical rotation direction was (+) (110 mg, optical purity 99.1% ee), and an enantiomer form (Example 4-2) in which retention time was long and the optical rotation direction was (−) (110 mg, optical purity 99.1% ee) were obtained as white powders, respectively.

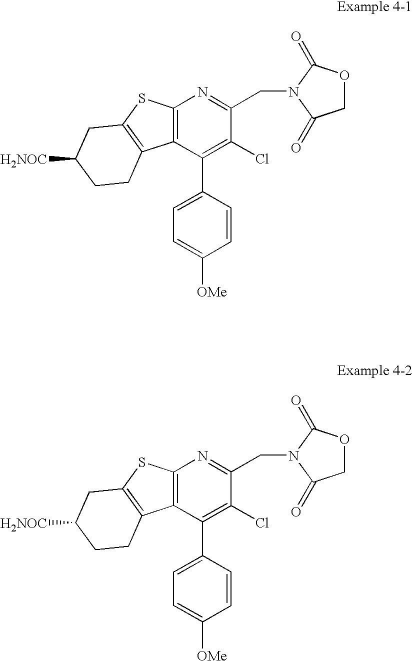

### Example 5

According to a method similar to that of Example 4, the compound obtained in Example 2 (3-chloro-2-[(2,5-dioxo-1-pyrrolidinyl)methyl]-5,6,7,8-tetrahydro-4-(4-methoxyphenyl)[1]benzothieno[2,3-b]pyridine-7-carboxamide, 991 mg) was fractionated to give an enantiomer form (Example 5-1) in which retention time was short and the optical rotation direction was (+) (490 mg, optical purity 99.9% ee), and an enantiomer form (Example 5-2) in which retention time was long and the optical rotation direction was (−) (480 mg, optical purity 99.8% ee) as white powders, respectively.

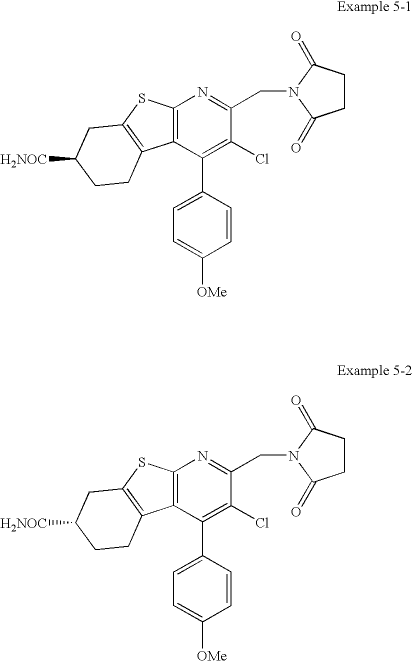

### Example 6

According to a method similar to that of Example 4, the compound obtained in Example 3 (3-chloro-2-[(2,4-dioxo-1,3-thiazolidin-3-yl)methyl]-5,6,7,8-tetrahydro-4-(4-methoxyphenyl)[1]benzothieno[2,3-b]pyridine-7-carboxamide, 997 mg) was fractionated to give an enantiomer form (Example 6-1) in which retention time was short and the optical rotation direction was (+) (495 mg, optical purity 99.8% ee), and an enantiomer form (Example 6-2) in which retention time was long and the optical rotation direction was (−) (498 mg, optical purity 99.8% ee) as white powders, respectively.

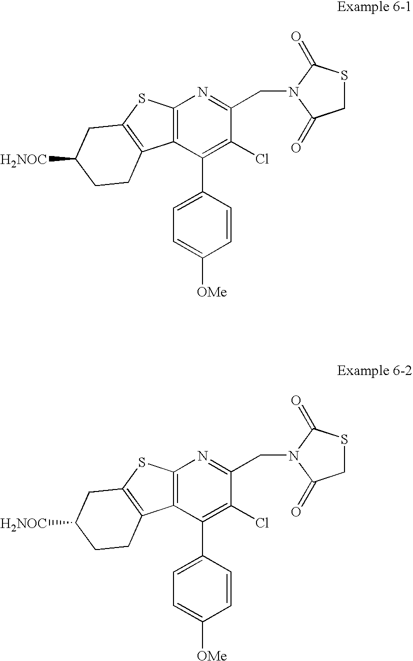

### Example 7

### (7S)-3-Chloro-2-[(2,4-dioxo-1,3-oxazolidin-3-yl)methyl]-5,6,7,8-tetrahydro-4-(4-hydroxyphenyl)[1]benzothieno[2,3-b]pyridine-7-carboxamide

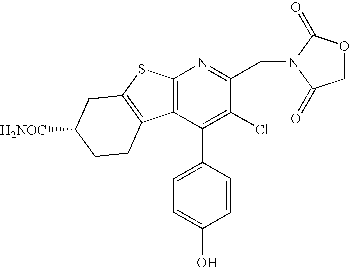

A mixture of (7S)-3-chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxamide (compound of Example 4-2, 133 mg), DL-methionine (123 mg) and methanesulfonic acid (4 ml) was stirred with heating at 80° C. for 3 hrs. The reaction solution was cooled and poured into water, and extracted with ethyl acetate. The ethyl acetate layer was washed with water and saturated brine and dried over magnesium sulfate, and the solvent was removed by evaporation under reduced pressure to give the title compound as colorless crystals (68 mg, 53%), which were recrystallized from THF-diethyl ether. Melting point: 317-318° C.

¹H-NMR (DMSO-d₆) δ; 1.38-1.61 (1H, m), 1.65-2.10 (3H, m), 2.89-2.99 (2H, m), 4.91 (2H, m), 5.14 (2H, s), 6.80-6.98 (3H, m), 7.02-7.22 (2H, m), 7.32 (1H, brs), 9.76 (1H, s).

Optical purity: 99.9% ee.

The compounds as shown in Examples 8 and 9 were synthesized according to a method similar to that of Example 7.

### Example 8

### (7S)-3-Chloro-2-[(2,5-dioxo-1-pyrrolidinyl)methyl]-5,6,7,8-tetrahydro-4-(4-hydroxyphenyl)[1]benzothieno[2,3-b]pyridine-7-carboxamide

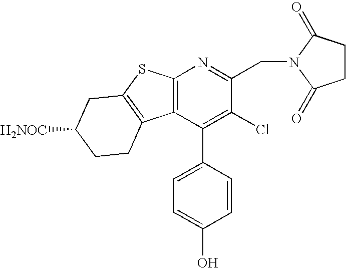

Melting point: 299-300° C. (solvent for recrystallization: THF-hexane).

### Example 9

### (7S)-3-Chloro-2-[(2,4-dioxo-1,3-thiazolidin-3-yl)methyl]-5,6,7,8-tetrahydro-4-(4-hydroxyphenyl)[1]benzothieno[2,3-b]pyridine-7-carboxamide

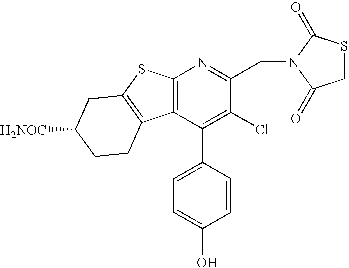

Melting point: 282-283° C. (solvent for recrystallization: THF-diethyl ether).

### Example 10

### (7S)-3-Chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxamide

(7S)-3-Chloro-2-[(2,4-dioxo-1,3-oxazolidin-3-yl)methyl]-5,6,7,8-tetrahydro-4-(4-methoxyphenyl)-N-[(1R)-1-phenylethyl][1]benzothieno[2,3-b]pyridine-7-carboxamide (1.0 g), anisole (0.2 ml) and methanesulfonic acid (2.5 ml) were mixed and stirred at 80° C. for 4 hrs. The mixture was cooled, and ethyl acetate (20 ml) and water (10 ml) were added dropwise. The organic layer was separated, washed with 5% aqueous sodium hydrogen carbonate and 10% brine (each 5 ml) and concentrated under reduced pressure. Ethyl acetate (10 ml) and n-heptane (10 ml) were added, and the precipitated crystals were collected by filtration and washed with ethyl acetate-n-heptane (1:1, 5 ml) to give the title compound as white crystals (0.69 g, yield 83.5%).

¹H-NMR (300 MHz, DMSO-d₆) δ; 1.43-1.54 (1H, m), 1.76-1.97 (3H, m) 2.49-2.56 (1H, m), 2.91-2.93 (2H, m), 3.85 (3H, s), 4.92 (2H, s), 5.14 (2H, s), 6.86 (1H, m), 7.05-7.11 (2H, m), 7.22-7.32 (3H, m).

### Example 11

### (7S)-3-Chloro-2-[(2,5-dioxo-1-pyrrolidinyl)methyl]-5,6,7,8-tetrahydro-4-(4-methoxyphenyl)[1]benzothieno[2,3-b]pyridine-7-carboxamide

(7S)-3-Chloro-2-[(2,5-dioxo-1-pyrrolidinyl)methyl]-5,6,7,8-tetrahydro-4-(4-methoxyphenyl)-N-[(1R)-1-phenylethyl][1]benzothieno[2,3-b]pyridine-7-carboxamide (1.0 g), anisole (0.2 ml) and methanesulfonic acid (2.5 ml) were mixed, and the mixture was stirred at 80° C. for 4 hrs. The mixture was cooled, and ethyl acetate (20 ml) and water (10 ml) were added dropwise. The organic layer was separated, washed with 5% aqueous sodium hydrogen carbonate and 10% brine (each 5 ml) and concentrated under reduced pressure. Ethyl acetate (10 ml) and n-heptane (10 ml) were added, and the precipitated crystals were collected by filtration and washed with ethyl acetate-n-heptane (1:1, 5 ml) to give the title compound as white crystals (0.73 g, yield 89.2%), which were recrystallized from ethyl acetate-n-heptane. Melting point: 282-283° C.

¹H-NMR (300 MHz, CDCl₃) δ; 1.62-1.97 (4H, m), 2.51-2.59 (1H, m), 2.90-3.06 (6H, m), 3.89 (3H, s), 5.01 (2H, s), 5.54 (2H, bs), 6.96-7.09 (3H, m), 7.17-7.21 (1H, m).

### Example 12

### (7S)-3-Chloro-2-[(2,4-dioxo-1,3-thiazolidin-3-yl)methyl]-5,6,7,8-tetrahydro-4-(4-methoxyphenyl)[1]benzothieno[2,3-b]pyridine-7-carboxamide

(7S)-3-Chloro-2-[(2,4-dioxo-1,3-thiazolidin-3-yl)methyl]-5,6,7,8-tetrahydro-4-(4-methoxyphenyl)-N-[(1R)-1-phenylethyl][1]benzothieno[2,3-b]pyridine-7-carboxamide (1.0 g), anisole (0.2 ml) and methanesulfonic acid (2.5 ml) were mixed, and the mixture was stirred at 80° C. for 4 hrs. The mixture was cooled, and ethyl acetate (20 ml) and water (10 ml) were added dropwise. The organic layer was separated, washed with 5% aqueous sodium hydrogen carbonate and 10% brine (each 5 ml) and concentrated under reduced pressure. The residue was purified by silica gel column chromatography eluting with ethyl acetate-n-hexane [3:1-1:0] to give the title compound as pale-brown crystals (0.66 g, yield 80%), which were recrystallized from ethyl acetate-n-hexane. Melting point: 145-148° C.

¹H-NMR (300 MHz, CDCl₃) δ; 1.63-1.98 (4H, m), 2.51-2.60 (1H, m) 2.96-3.09 (2H, m), 3.89 (3H, s), 4.15 (2H, s), 5.11 (2H, s), 5.57 (2H, bs), 6.96-7.10 (3H, m), 7.18-7.21 (1H, m).

### Example 13

### (7S)-3-Chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxamide

(7S)-3-Chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxylic acid (0.50 g), 1-hydroxybenzotriazole ammonium salt (164.4 mg) and WSC (207.1 mg) were added to DMF (2.5 ml), and the mixture was stirred at room temperature for 1.5 hrs. Ethyl acetate (7.5 ml) and water (5 ml) were added, and the organic layer was separated and washed with 10% brine. The solvent was evaporated, and the residue was dissolved in ethyl acetate (3 ml) in an outer bath at 50 to 55° C. n-Hexane (1.5 ml) was added dropwise, and the mixture was stirred at the same temperature for 30 min, and at room temperature for 1 hr. The precipitated crystals were collected by filtration and washed with ethyl acetate-n-hexane=1:1 to give the title compound. Yield: 0.36 g (72.15%). The crystals were recrystallized from methyl ethyl ketone. Melting point: 229-231° C.

Optical purity: 95.00% ee

Conditions for Measurement of Optical Purity

Column: CHIRALCEL OJ-R

Mobile phase: 0.05 M KH₂PO₄: CH₃CN=7:3

Flow rate: 1.0 ml/min.

Temperature: 25° C.

Measurement wavelength: 262 nm

¹H-NMR (300 MHz, CDCl₃) δ; 1.61-1.75 (1H, m), 1.89-1.99 (3H, m) 2.54-2.59 (1H, m), 3.02-3.08 (2H, m), 3.90 (3H, s), 4.92 (2H, s), 5.05 (2H, s), 5.56 (2H, br), 6.97-7.03 (2H, m), 7.06-7.10 (1H, m), 7.18-7.21 (1H, m).

The title compound obtained by the above-mentioned method was suspended in 2-propanol at a concentration of 20 mg/20 ml at room temperature for 2 hrs, and the obtained precipitate was dried under a nitrogen stream to give crystals of the above-mentioned compound. The crystals were measured by DSC (DSC6200R, temperature rising rate: 5° C./min) and found to show a peak (melting point) at 178.2° C.

### Formulation Example 1

| | | |
|---|---|---|
| (1) | Compound of Example 1 | 10.0 g |
| (2) | Lactose | 70.0 g |
| (3) | Cornstarch | 50.0 g |
| (4) | Soluble starch | 7.0 g |
| (5) | Magnesium stearate | 3.0 g |

The compound of Example 1 (10.0 g) and magnesium stearate (3.0 g) are granulated with an aqueous solution of soluble starch (70 ml, 7.0 g as soluble starch), dried and mixed with lactose (70.0 g) and corn starch (50.0 g) (lactose, corn starch, soluble starch and magnesium stearate are all products on the Japanese Pharmacopoeia 14^{th} ed.). The mixture is compressed to give tablets.

### Formulation Example 2

The preparations were produced in the formulation systems shown in Table 1. To be specific, granule mixtures of Example compounds prepared by the fluidized bed granulating method (FD-5S, POWREX), mannitol, crystalline cellulose, hydroxypropyl cellulose (HPC-L), carboxymethyl cellulose calcium (ECG-505) and magnesium stearate were tableted by a tabletting machine (Correct 19K, Kikusui Seisakusho Ltd.) using a 9.2 mmφ pounder. A film coating liquid comprising hydroxypropylmethyl cellulose (TC-5R), polyethylene glycol 6000 (PEG 6000), titanium oxide and yellow diiron trioxide was sprayed on the obtained tablet using a pan coating machine (High Coater, Freund Corporation) to give film-coated tablets.

TABLE 1

| | Amounts of ingredients (mg) | | | | |
|---|---|---|---|---|---|
| | Placebo | 1 mg | 5 mg | 25 mg | 100 mg |
| | tablet | tablet | tablet | tablet | tablet |
| Example compound | 0.0 | 1.0 | 5.0 | 25.0 | 100.0 |
| Mannitol | 228.0 | 227.0 | 223.0 | 203.0 | 128.0 |
| Crystalline | 45.0 | 45.0 | 45.0 | 45.0 | 45.0 |
| cellulose | | | | | |
| Hydroxypropyl | 9.0 | 9.0 | 9.0 | 9.0 | 9.0 |
| cellulose (HPC-L) | | | | | |
| Carboxymethyl | 15.0 | 15.0 | 15.0 | 15.0 | 15.0 |
| cellulose calcium | | | | | |
| (ECG-505) | | | | | |
| Magnesium stearate | 3.0 | 3.0 | 3.0 | 3.0 | 3.0 |
| Naked tablet total | 300.0 | 300.0 | 300.0 | 300.0 | 300.0 |
| Hydroxypropylmethyl | 9.0 | 9.0 | 9.0 | 9.0 | 9.0 |
| cellulose (TC-5R) | | | | | |
| Polyethylene glycol | 1.8 | 1.8 | 1.8 | 1.8 | 1.8 |
| 6000 (PEG 6000) | | | | | |
| Titanium oxide | 1.2 | 1.2 | 1.2 | 1.2 | 1.2 |
| Yellow diiron | 0.04 | 0.04 | 0.04 | 0.04 | 0.04 |
| trioxide | | | | | |
| Film tablet total | 312.0 | 312.0 | 312.0 | 312.0 | 312.0 |

### Experimental Example 1

### Effect on Adjuvant Arthritis in Rat

A male Lewis rat (7-week-old, Crea Japan Ltd.) was sensitized by an intracutaneous injection of 0.05 ml of Freund's complete adjuvant: 0.5% liquid paraffin suspension of dead tubercle bacillus) into the footpad of the right hind paw, and the test compound suspended in 0.5% methyl cellulose was orally administered once a day for 14 days. Immediately before sensitization (Day 0) and 14th day (Day 14), the edema volume of the left hind paw was measured by a plethysmometer (manufactured by Ugo Basile, Italy) and the footpad swelling suppression rate (%) relative to unsensitized rat was determined by the following formula.

$$Footpad sweeling suppression rate ( \% ) = ( 1 - \frac{( \begin{matrix}foot edema volume \\ of drug administration \\ group\end{matrix} ) ( \begin{matrix}foot edema volume \\ of unsensitized group\end{matrix} )}{( \begin{matrix}foot edema volume \\ of drug non \text{-} administration \\ group\end{matrix} ) ( \begin{matrix}foot edema volume \\ of unsensitized group\end{matrix} )} ) \times 100$$

The results are shown in mean ±S.E. of each group (n=6) and compared and detected by the Dunnett's method. In addition, less than 5% of the critical rate was taken as significant. As shown in Table 2, the compound of the present invention showed an effect in the suppression of footpad edema.

TABLE 2

| Compound | Dose | Swelling suppression |
|---|---|---|
| (Ex. No.) | (mg/kg) | rate (%) |
| 4-2 | 0.3 | 82* |
| 5-2 | 0.3 | 88* |
| 6-2 | 0.3 | 87* |

### INDUSTRIAL APPLICABILITY

The compound (I) of the present invention is useful as an anti-inflammatory agent, particularly as a prophylactic or therapeutic agent for arthritis such as rheumatoid arthritis and the like, because it has a superior anti-inflammatory activity, useful for the prophylaxis or treatment of bone destruction, osteoporosis and the like associated with arthritis because it has a superior bone resorption suppressing activity, useful for the prophylaxis or treatment of a disease caused by immunity such as autoimmune diseases because it has a superior immune cytokine production-suppressing activity, and also useful as an agent for the prophylaxis or treatment of rejection after organ transplantation. Moreover, since compound (I) of the present invention is low toxic and superior in oral absorbability, and it exhibits efficacy for a long time, because it is stable against in vivo metabolism, the compound (I) can be advantageously used as a pharmaceutical agent.

This application is based on patent application Nos. 2004-144659 and 2005-073745 filed in Japan, the contents of which are hereby incorporated by reference. All of the references cited herein, including patents, patent applications and publications, are hereby incorporated in their entireties by reference.

1. A compound represented by the formula (I) 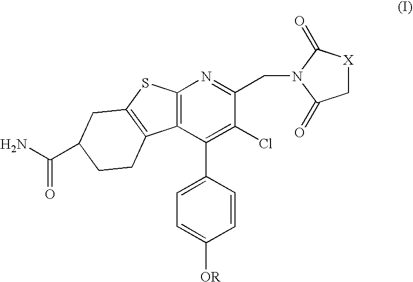
wherein R is a hydrogen atom or a C₁₋₄ alkyl group and X is CH₂, O or S
or a salt thereof.

2. 3-Chloro-2-[(2,4-dioxo-1,3-oxazolidin-3-yl)methyl]-5,6,7,8-tetrahydro-4-(4-methoxyphenyl)[1]benzothieno[2,3-b]pyridine-7-carboxamide or a salt thereof,
3-chloro-2-[(2,5-dioxo-1-pyrrolidinyl)methyl]-5,6,7,8-tetrahydro-4-(4-methoxyphenyl)[1]benzothieno[2,3-b]pyridine-7-carboxamide or a salt thereof, or
3-chloro-2-[(2,4-dioxo-1,3-thiazolidin-3-yl)methyl]-5,6,7,8-tetrahydro-4-(4-methoxyphenyl)[1]benzothieno[2,3-b]pyridine-7-carboxamide, or a salt thereof.

3. A prodrug of the compound of claim 1.

4. A production method of an optically active compound represented by the formula 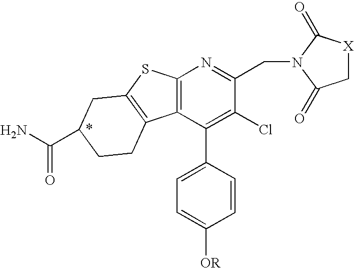
wherein R is a hydrogen atom or a C₁₋₄ alkyl group, X is CH₂, O or S and * shows the position of the optically active asymmetric carbon, or a salt thereof,
which comprises cleaving an amino-protecting group of an optically active compound represented by the formula 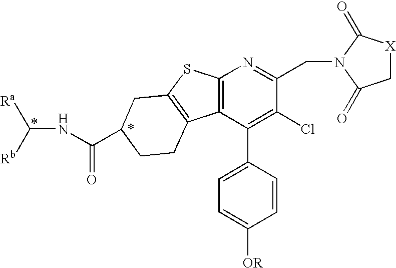
wherein R^{a} is an aryl group optionally having substituents, R^{b} is an alkyl group optionally having substituents or an aryl group optionally having substituents, which is different from R^{a}, and other symbols are as defined above, or a salt thereof.

5. The production method of claim 4, wherein R^{a} is a phenyl group optionally having substituents, R^{b} is an alkyl group optionally having substituents or a phenyl group optionally having substituents, which is different from R^{a}.

6. A production method of an optically active compound represented by the formula 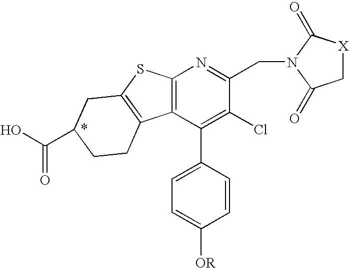
wherein R is a hydrogen atom or a C₁₋₄ alkyl group, X is CH₂, O or S and * shows the position of the optically active asymmetric carbon, or a salt thereof,
which comprises hydrolyzing an optically active compound represented by the formula 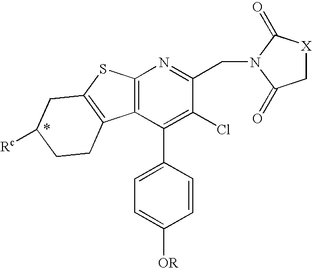
wherein R^{c} is an ester group having an optically active asymmetric carbon, and other symbols are as defined above, or a salt thereof.

7. A production method of an optically active compound represented by the formula 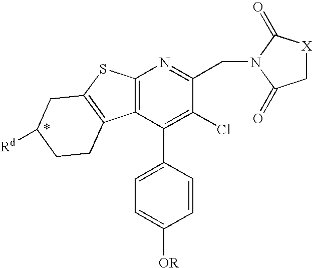
wherein R is a hydrogen atom or a C₁₋₄ alkyl group, R^{d} is an esterified or amidated carboxyl group having an optically active asymmetric carbon, X is CH₂, O or S, and * shows the position of the optically active asymmetric carbon, or a salt thereof, which comprises reacting a compound represented by the formula 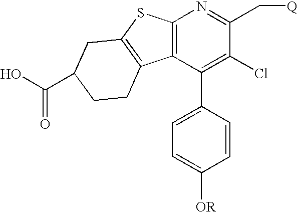
wherein R is as defined above and Q is a leaving group or a group represented by the formula 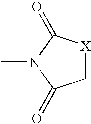
wherein X is as defined above, or a salt thereof, with an amine compound having an optically active asymmetric carbon or an alcohol compound having an optically active asymmetric carbon, or a salt-thereof to give a diastereomer mixture or a salt thereof, optically resolving the diastereomer mixture or a salt thereof, and when Q is a leaving group, further reacting with a compound represented by the formula 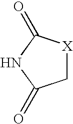
wherein X is as defined above, or a salt thereof.

8. A pharmaceutical agent comprising the compound of claim 1 or a prodrug thereof.

9. The pharmaceutical agent of claim 8, which is a T cell differentiation modulating agent.

10. The pharmaceutical agent of claim 8, which is an agent for the prophylaxis or treatment of inflammatory diseases.

11. The pharmaceutical agent of claim 8, which is an agent for the prophylaxis or treatment of immune diseases.

12. The pharmaceutical agent of claim 8, which is an agent for the prophylaxis or treatment of rheumatoid arthritis.

13. A method for preventing or treating rheumatoid arthritis, which comprises administering an effective amount of the compound of claim 1 or a prodrug thereof to a mammal.

14. Use of the compound of claim 1 or a prodrug thereof for the production of an agent for the prophylaxis or treatment of rheumatoid arthritis.

15. A compound represented by the formula 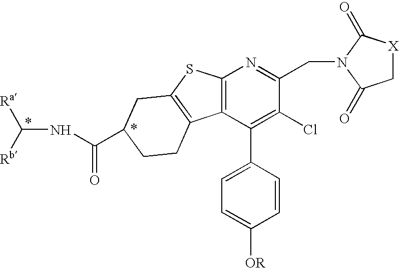
wherein R is a hydrogen atom or a C₁₋₄ alkyl group, R^{a′} is an aryl group optionally having substituents, R^{b′} is an alkyl group optionally having substituents, X is CH₂, O or S, and * shows the position of the optically active asymmetric carbon, or a salt thereof.

16. A compound represented by the formula 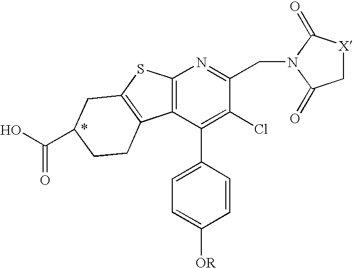
wherein R is a hydrogen atom or a C₁₋₄ alkyl group, X′ is O or S, and * shows the position of the optically active asymmetric carbon, or a salt thereof.

17. A compound represented by the formula 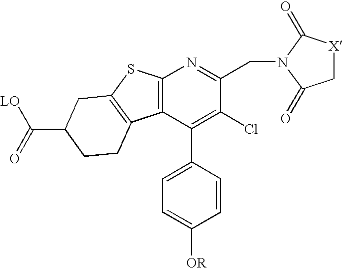
wherein R is a hydrogen atom or a C₁₋₄ alkyl group, X′ is O or S, and L is a C₁₋₄ alkyl group, or a salt thereof.
